SEQUENCE LISTING (73 sequences)

SEQ ID NO: 1 - protein, 94 aa, Homo sapiens
VSDVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 2 - protein, 7 aa, synthetic construct
  UNSURE 1..7: note = modified BC loop region of NRAS-binding monobody
  UNSURE 7: note = X at position 7 is any amino acid residue, preferably Asp or Glu
APAVTVX

SEQ ID NO: 3 - protein, 7 aa, synthetic construct
  UNSURE 1..7: note = modified BC loop region of NRAS-binding monobody
APAVTVD

SEQ ID NO: 4 - skipped

SEQ ID NO: 5 - protein, 7 aa, synthetic construct
  UNSURE 1..7: note = modified CD loop region of NRAS-binding monobody
GSYYPYA

SEQ ID NO: 6 - protein, 7 aa, synthetic construct
  UNSURE 1..7: note = modified CD loop region of NRAS-binding monobody
GAYYPYA

SEQ ID NO: 7 - protein, 7 aa, synthetic construct
  UNSURE 1..7: note = modified CD loop region of NRAS-binding monobody
GGYAWAY

SEQ ID NO: 8 - skipped

SEQ ID NO: 9 - protein, 5 aa, synthetic construct
  UNSURE 1..5: note = wildtype DE loop region of NRAS-binding monobody
PGSKS

SEQ ID NO: 10 - skipped

SEQ ID NO: 11 - protein, 12 aa, synthetic construct
  UNSURE 1..12: note = modified FG loop region of NRAS-binding monobody
YQVIGAIWKYYS

SEQ ID NO: 12 - protein, 95 aa, synthetic construct
  UNSURE 1..95: note = NRAS-binding monobody named Mb(NRAS_19)
VSSVPTKLEVVAATPTSLLISWDAPAVTVDFYVITYGETGSYYPYAQEFKVPGSKSTATISGLSPGVDYTITVYAYQVIGAIWKYYSPISINYRT

SEQ ID NO: 13 - protein, 95 aa, synthetic construct
  UNSURE 1..95: note = NRAS-binding monobody named Mb(NRAS_20)
VSSVPTKLEVVAATPTSLLISWDAPAVTVDFYVITYGETGAYYPYAQEFKVPGSKSTATISGLSPGVDYTITVYAYQVIGAIWKYYSPISINYRT

SEQ ID NO: 14 - protein, 95 aa, synthetic construct
  UNSURE 1..95: note = NRAS-binding monobody named Mb(NRAS_24)
VSSVPTKLEVVAATPTSLLISWDAPAVTVDFYVITYGETGGYAWAYQEFKVPGSKSTATISGLSPGVDYTITVYAYQVIGAIWKYYSPISINYRT

SEQ ID NO: 15 - protein, 11 aa, synthetic construct
  UNSURE 1..11: note = modified FG loop region of NRAS-binding monobody
SQVIGAIWKYY

SEQ ID NO: 16 - protein, 11 aa, synthetic construct
  UNSURE 1..11: note = modified FG loop region of NRAS-binding monobody
YHVIGAIWKYY

SEQ ID NO: 17 - protein, 11 aa, synthetic construct
  UNSURE 1..11: note = modified FG loop region of NRAS-binding monobody
YQIDGAIWKYY

SEQ ID NO: 18 - protein, 11 aa, synthetic construct
  UNSURE 1..11: note = modified FG loop region of NRAS-binding monobody
YQIEGAIWKYY

SEQ ID NO: 19 - protein, 11 aa, synthetic construct
  UNSURE 1..11: note = modified FG loop region of NRAS-binding monobody
YQIHGAIWKYY

SEQ ID NO: 20 - protein, 11 aa, synthetic construct
  UNSURE 1..11: note = modified FG loop region of NRAS-binding monobody
YQIKGAIWKYY

SEQ ID NO: 21 - protein, 11 aa, synthetic construct
  UNSURE 1..11: note = modified FG loop region of NRAS-binding monobody
YQILGAIWKYY

SEQ ID NO: 22 - protein, 11 aa, synthetic construct
  UNSURE 1..11: note = modified FG loop region of NRAS-binding monobody
YQIRGAIWKYY

SEQ ID NO: 23 - protein, 11 aa, synthetic construct
  UNSURE 1..11: note = modified FG loop region of NRAS-binding monobody
YQIVGAIWKYY

SEQ ID NO: 24 - protein, 11 aa, synthetic construct
  UNSURE 1..11: note = modified FG loop region of NRAS-binding monobody
YQIYGAIWKYY

SEQ ID NO: 25 - protein, 11 aa, synthetic construct
  UNSURE 1..11: note = modified FG loop region of NRAS-binding monobody
YQLDGAIWKYY

SEQ ID NO: 26 - protein, 11 aa, synthetic construct
  UNSURE 1..11: note = modified FG loop region of NRAS-binding monobody
YQLEGAIWKYY

SEQ ID NO: 27 - protein, 11 aa, synthetic construct
  UNSURE 1..11: note = modified FG loop region of NRAS-binding monobody
YQLHGAIWKYY

SEQ ID NO: 28 - protein, 11 aa, synthetic construct
  UNSURE 1..11: note = modified FG loop region of NRAS-binding monobody
YQLLGAIWKYY

SEQ ID NO: 29 - protein, 11 aa, synthetic construct
  UNSURE 1..11: note = modified FG loop region of NRAS-binding monobody
YQLYGAIWKYY

SEQ ID NO: 30 - protein, 11 aa, synthetic construct
  UNSURE 1..11: note = modified FG loop region of NRAS-binding monobody
YQTDGAIWKYY

SEQ ID NO: 31 - protein, 11 aa, synthetic construct
  UNSURE 1..11: note = modified FG loop region of NRAS-binding monobody
YQTEGAIWKYY

SEQ ID NO: 32 - protein, 11 aa, synthetic construct
  UNSURE 1..11: note = modified FG loop region of NRAS-binding monobody
YQTHGAIWKYY

SEQ ID NO: 33 - protein, 11 aa, synthetic construct
  UNSURE 1..11: note = modified FG loop region of NRAS-binding monobody
YQTLGAIWKYY

SEQ ID NO: 34 - protein, 11 aa, synthetic construct
  UNSURE 1..11: note = modified FG loop region of NRAS-binding monobody
YQTVGAIWKYY

SEQ ID NO: 35 - protein, 11 aa, synthetic construct
  UNSURE 1..11: note = modified FG loop region of NRAS-binding monobody
YQTYGAIWKYY

SEQ ID NO: 36 - protein, 11 aa, synthetic construct
  UNSURE 1..11: note = modified FG loop region of NRAS-binding monobody
YQVIAHIWKYY

SEQ ID NO: 37 - protein, 11 aa, synthetic construct
  UNSURE 1..11: note = modified FG loop region of NRAS-binding monobody
YQVIASIWKYY

SEQ ID NO: 38 - protein, 11 aa, synthetic construct
  UNSURE 1..11: note = modified FG loop region of NRAS-binding monobody
YQVIAYIWKYY

SEQ ID NO: 39 - protein, 11 aa, synthetic construct
  UNSURE 1..11: note = modified FG loop region of NRAS-binding monobody
YQVIDHIWKYY

SEQ ID NO: 40 - protein, 11 aa, synthetic construct
  UNSURE 1..11: note = modified FG loop region of NRAS-binding monobody
YQVIDSIWKYY

SEQ ID NO: 41 - protein, 11 aa, synthetic construct
  UNSURE 1..11: note = modified FG loop region of NRAS-binding monobody
YQVIDYIWKYY

SEQ ID NO: 42 - protein, 11 aa, synthetic construct
  UNSURE 1..11: note = modified FG loop region of NRAS-binding monobody
YQVIEHIWKYY

SEQ ID NO: 43 - protein, 11 aa, synthetic construct
  UNSURE 1..11: note = modified FG loop region of NRAS-binding monobody
YQVIESIWKYY

SEQ ID NO: 44 - protein, 11 aa, synthetic construct
  UNSURE 1..11: note = modified FG loop region of NRAS-binding monobody
YQVIEYIWKYY

SEQ ID NO: 45 - protein, 11 aa, synthetic construct
  UNSURE 1..11: note = modified FG loop region of NRAS-binding monobody
YQVIGAICKYY

SEQ ID NO: 46 - protein, 11 aa, synthetic construct
  UNSURE 1..11: note = modified FG loop region of NRAS-binding monobody
YQVIGALWKYY

SEQ ID NO: 47 - protein, 11 aa, synthetic construct
  UNSURE 1..11: note = modified FG loop region of NRAS-binding monobody
YQVIHHIWKYY

SEQ ID NO: 48 - protein, 11 aa, synthetic construct
  UNSURE 1..11: note = modified FG loop region of NRAS-binding monobody
YQVIHSIWKYY

SEQ ID NO: 49 - protein, 11 aa, synthetic construct
  UNSURE 1..11: note = modified FG loop region of NRAS-binding monobody
YQVIHYIWKYY

SEQ ID NO: 50 - protein, 11 aa, synthetic construct
  UNSURE 1..11: note = modified FG loop region of NRAS-binding monobody
YQVIKHIWKYY

SEQ ID NO: 51 - protein, 11 aa, synthetic construct
  UNSURE 1..11: note = modified FG loop region of NRAS-binding monobody
YQVIKSIWKYY

SEQ ID NO: 52 - protein, 11 aa, synthetic construct
  UNSURE 1..11: note = modified FG loop region of NRAS-binding monobody
YQVIKYIWKYY

SEQ ID NO: 53 - protein, 11 aa, synthetic construct
  UNSURE 1..11: note = modified FG loop region of NRAS-binding monobody
YQVILHIWKYY

SEQ ID NO: 54 - protein, 11 aa, synthetic construct
  UNSURE 1..11: note = modified FG loop region of NRAS-binding monobody
YQVIRHIWKYY

SEQ ID NO: 55 - protein, 11 aa, synthetic construct
  UNSURE 1..11: note = modified FG loop region of NRAS-binding monobody
YQVIRSIWKYY

SEQ ID NO: 56 - protein, 11 aa, synthetic construct
  UNSURE 1..11: note = modified FG loop region of NRAS-binding monobody
YQVIRYIWKYY

SEQ ID NO: 57 - protein, 11 aa, synthetic construct
  UNSURE 1..11: note = modified FG loop region of NRAS-binding monobody
YQVISHIWKYY

SEQ ID NO: 58 - protein, 11 aa, synthetic construct
  UNSURE 1..11: note = modified FG loop region of NRAS-binding monobody
YQVISSIWKYY

SEQ ID NO: 59 - protein, 11 aa, synthetic construct
  UNSURE 1..11: note = modified FG loop region of NRAS-binding monobody
YQVISYIWKYY

SEQ ID NO: 60 - protein, 11 aa, synthetic construct
  UNSURE 1..11: note = modified FG loop region of NRAS-binding monobody
YQVITHIWKYY

SEQ ID NO: 61 - protein, 11 aa, synthetic construct
  UNSURE 1..11: note = modified FG loop region of NRAS-binding monobody
YQVIYHIWKYY

SEQ ID NO: 62 - protein, 11 aa, synthetic construct
  UNSURE 1..11: note = modified FG loop region of NRAS-binding monobody
YQVIYYIWKYY

SEQ ID NO: 63 - protein, 11 aa, synthetic construct
  UNSURE 1..11: note = modified FG loop region of NRAS-binding monobody
YQVSGAIWKYY

SEQ ID NO: 64 - protein, 11 aa, synthetic construct
  UNSURE 1..11: note = modified FG loop region of NRAS-binding monobody
YQYEGAIWKYY

SEQ ID NO: 65 - protein, 11 aa, synthetic construct
  UNSURE 1..11: note = modified FG loop region of NRAS-binding monobody
YQYHGAIWKYY

SEQ ID NO: 66 - protein, 11 aa, synthetic construct
  UNSURE 1..11: note = modified FG loop region of NRAS-binding monobody
YQYVGAIWKYY

SEQ ID NO: 67 - protein, 11 aa, synthetic construct
  UNSURE 1..11: note = modified FG loop region of NRAS-binding monobody
YQYYGAIWKYY

SEQ ID NO: 68 - protein, 95 aa, synthetic construct
  UNSURE 1..11: note = NRAS-binding monobody named Mb(NRAS_9)
VSSVPTKLEVVAATPTSLLISWDAPAVTVDFYVITYGETGSYYPYAQEFKVPGSKSTATISGLKPGVDYTITVYAYQVIGAIWKYYSPISINYRT

SEQ ID NO: 69 - protein, 97 aa, synthetic construct
  UNSURE 1..97: note = consensus for NRAS-binding monobody library structure
  UNSURE 30: note = X at position 30 is any of Asn, Asp, His, Ile, Leu, Phe, Tyr and Val
  UNSURE 31: note = X at position 31 is any of His, Leu, Phe and Tyr
  UNSURE 33: note = X at position 33 is any of Asn, Asp, His, Ile, Leu, Phe, Tyr and Val
  UNSURE 41..45: note = X at positions 41-45 is any amino acid except Cys
  UNSURE 46: note = X at position 46 is optional, and is any amino acid except Cys
  UNSURE 48: note = X at position 48 is any of Ala, Glu, Lys and Thr
  UNSURE 50: note = X at position 50 is any of Ala, Glu, Lys and Thr
  UNSURE 76..82: note = X at positions 76-82 is any amino acid except Cys
  UNSURE 83..88: note = X at positions 83-88 is optional, and is any amino acid except Cys
VSSVPTKLEVVAATPTSLLISWDAPAVTVXXYXITYGETGXXXXXXQXFXVPGSKSTATISGLSPGVDYTITVYAXXXXXXXXXXXXXSPISINYRT

SEQ ID NO: 70 - DNA, 288 bp, synthetic construct
  source 1..288: mol_type = other DNA
  unsure 1..288: note = DNA molecule encoding NRAS-binding monobody named Mb(NRAS_19)
gtgtccagcgtgcccaccaagctggaagtggtcgccgctacacctacaagcctgctgatctcttgggacgcccctgctgtgaccgtggacttctacgtgatcacctacggcgagacaggcagctactatccttacgcccaggagttcaaggtgccaggatctaaaagcaccgccaccatctccggcctgagccctggcgtggattacaccattacagtgtacgcctaccaggtgatcggcgccatctggaagtactacagccccatcagcatcaactacagaacctaa

SEQ ID NO: 71 - DNA, 288 bp, synthetic construct
  source 1..288: mol_type = other DNA
  unsure 1..288: note = DNA molecule encoding NRAS-binding monobody named Mb(NRAS_20)
gtttctagcgtgcctacaaagctggaagtggtcgccgccacccctaccagcctgctgatcagctgggacgcccctgccgtgaccgtggacttctacgtgatcacctacggcgagacaggcgcttattacccctacgcccaggagttcaaggtgccaggcagcaagtccaccgccacaatctccggcctgagccctggcgtggattacaccatcacagtgtacgcctaccaggtgattggagctatctggaaatactacagccccatctctatcaactacagaacctaa

SEQ ID NO: 72 - DNA, 288 bp, synthetic construct
  source 1..288: mol_type = other DNA
  unsure 1..288: note = DNA molecule encoding NRAS-binding monobody named Mb(NRAS_24)
gtgtccagcgtgcccacaaagctggaagtggttgccgctacacctaccagcctgctgatctcttgggacgcccctgccgtgaccgtggacttctacgtgattacctacggcgagacaggcggatacgcctgggcttatcaggagttcaaggtgcctggatctaaaagcaccgccacaatctccggcctgagccccggcgtggattacaccatcaccgtctacgcctaccaggtgatcggcgccatctggaagtactacagcccaatcagcatcaactacagaacctaa

SEQ ID NO: 73 - DNA, 288 bp, synthetic construct
  source 1..288: mol_type = other DNA
  unsure 1..288: note = DNA molecule encoding NRAS-binding monobody named Mb(NRAS_9)
gtttccagcgtgcctaccaagctggaagtggtggccgccacccctacaagcctgctgatcagctgggacgcccctgctgtgaccgtggatttctacgtgattacatacggcgagacaggatcttattacccctacgcccaggagttcaaggtgccaggctctaaaagcaccgccacaatctccggcctgaagcctggcgtggactacaccatcaccgtctacgcctaccaggtgatcggcgctatctggaagtactacagccccatcagcatcaactacagaacctaa

(19) United States
(12) Patent Application Publication
(10) Pub. No.: US 2026/0263609 A1
(43) Pub. Date: Sep. 10, 2026

(54) MONOBODIES SELECTIVE TO NRAS

(71) Applicant: NEW YORK UNIVERSITY, New York, NY (US)

(72) Inventors: Shohei KOIDE, New York, NY (US); Akiko KOIDE, New York, NY (US)

(21) Appl. No.: 18/848,985

(22) PCT Filed: Mar. 29, 2023
(86) PCT No.: PCT/US2023/065114

§ 371 Date: Sep. 20, 2024

Related U.S. Application Data
(60) Provisional application No. 63/325,144, filed on Mar. 29, 2022.

(51) Int. Cl. A61K 47/64; A61K 38/00; C07K 14/78; C12N 15/86; C12N 15/88; G01N 33/542

(52) U.S. Cl. CPC A61K 47/64; C07K 14/78; C12N 15/86; C12N 15/88; G01N 33/542; A61K 38/00; C07K 2319/10; C07K 2319/55; C07K 2319/60; C12N 2710/24143; C12N 2740/10043; C12N 2740/15043; C12N 2750/14143

(57) ABSTRACT

The present application relates to Neuroblastoma-RAS (“NRAS”) binding polypeptides. These binding polypeptide comprise a fibronectin type III (FN3) domain having one or more modified loop sequences include a modified FG loop amino acid sequence, a modified BC loop amino acid sequence, a modified CD loop amino acid sequence, a modified DE loop amino acid sequence, or a combination thereof, and optionally one or more modified beta strand sequences, wherein the one or more modified loop sequences and the one or more optional modified beta strand sequences enable selective binding to NRAS but not KRAS or HRAS. Also disclosed are conjugates that include the NRAS binding polypeptide, polynucleotides encoding the same, and methods of using these materials for treating cancer as well as screening for other agents capable of binding NRAS.

This application claims the priority benefit of U.S. Provisional Patent Application Ser. No. 63/325,144, filed Mar. 29, 2022, which is hereby incorporated by reference in its entirety.

### SEQUENCE LISTING

The Sequence Listing is being submitted electronically in XML format and is hereby incorporated by reference in its entirety. Said XML copy, created on Mar. 28, 2023, is named 147462002371.xml and is 94.0 KB in Size. No new matter is being introduced

### FIELD OF THE DISCLOSURE

The present invention relates generally to Neuroblastoma-RAS (“NRAS”) binding polypeptides and NRAS binding polypeptide conjugates; polynucleotides encoding either the NRAS binding polypeptides or NRAS binding polypeptide conjugates; and various methods of using the same for the treatment of oncogenic NRAS-mediated cancers.

### BACKGROUND

Activating, missense mutations of RAS genes (Neuroblastoma-RAS or NRAS; Harvey RAS or HRAS; and Kirsten RAS or KRAS) are frequently associated with human cancers, and they play important roles in oncogenic transformation (Cox et al., “Drugging the Undruggable RAS: Mission Possible? Nat Rev Drug Discov 13:828-851 (2014) (PMID: 25323927); Moore et al., “RAS-targeted Therapies: Is the Undruggable Drugged?” Nat Rev Drug Discov 19:533-552 (2020) (PMID: 32528145)). Oncogenic RAS mutant proteins have long been considered undruggable, due to an apparent lack of binding pockets suitable for small molecule inhibitors and the minute differences of RAS mutants from the wild-type proteins. Mutations of NRAS are particularly associated with a subset of human cancers, including skin cutaneous melanoma, multiple myeloma, acute myeloid leukemia, thyroid carcinoma and colorectal adenocarcinoma (Cox et al., “Drugging the Undruggable RAS: Mission Possible? Nat Rev Drug Discov 13:828-851 (2014) (PMID: 25323927); AACR Project GENIE Consortium, “AACR Project GENIE: Powering Precision Medicine Through an International Consortium,” Cancer Discovery 7 (8): 818-831 (2017)). Consequently, there may be therapeutic opportunities for these cancers with inhibitors with specificity to NRAS, but not selective to a specific oncogenic mutation. Such inhibitors would not affect normal functions mediated by the wild-type (unmutated) forms of the other RAS isoforms, KRAS and HRAS, which in turn would minimize undesirable side effects by sparing many crucial signaling events mediated by wild-type.

Presently, there are no reports of direct inhibitors selective to NRAS among RAS isotypes. Indeed, one report regarding inhibition of NRAS signaling describe inhibition of an upstream activator of NRAS, STK19 Kinase (Qian et al., “Targeting NRAS-Mutant Cancers with the Selective STK19 Kinase Inhibitor Chelidonine,” Clin Cancer Res. 26 (13): 3408-3419 (2020) (doi: 10.1158/1078-0432.CCR-19-2604; PMID: 32156748). Most other research has focused on downstream effectors of the MAPK pathway with MEK 1/2 inhibitors (see Garcia-Alvarez et al., “Current Perspectives and Novel Strategies of NRAS-Mutant Melanoma,” Onco Targets Ther. 14:3709-3719 (2021) (doi: 10.2147/OTT.S278095)).

A monobody termed NS1 was previously shown to inhibit signaling and tumorigenesis mediated by KRAS and HRAS, but not by NRAS, when expressed intracellularly as a genetically encoded reagent (Spencer-Smith et al., “Inhibition of RAS Function Through Targeting an Allosteric Regulatory Site,” Nat Chem Biol. 13 (1): 62-8 (2017) (doi: 10.1038/nchembio.2231; PMID: 27820802); Khan et al., “Targeting the alpha4-alpha5 Dimerization Interface of K-RAS Inhibits Tumor Formation in vivo,” Oncogene 38 (16): 2984-93 (2019) (doi: 10.1038/s41388-018-0636-y; PMID: 30573767)). Monobodies are synthetic binding proteins constructed on the molecular scaffold of a fibronectin type III domain (FN3) (Koide et al., “The Fibronectin Type III Domain as a Scaffold for Novel Binding Proteins,” J Mol Biol. 284:1141-51 (1998); Hantschel et al., “Monobodies as Enabling Tools for Structural and Mechanistic Biology,” Curr Opin Struct Biol. 60:167-74 (2020) (doi: 10.1016/j.sbi.2020.01.015; PMID: 32145686); Akkapeddi et al., “Monobodies as Tool Biologics for Accelerating Target Validation and Druggable Site Discovery,” RSC Med Chem. 12 (11): 1839-53 (2021) (doi: 10.1039/d1md00188d; PMID: 34820623)). The crystal structure of NS1 in complex with HRAS revealed that NS1 binds to the alpha4-alpha5 region of RAS distant from the so-called Switch region. The Switch region had been known to be important for RAS function and oncogenic RAS mutations are located within or close to the Switch region. Thus, targeting the Switch region has thus far been the primary focus of RAS inhibitor development.

NS1 inhibits RAS-mediated functions in a distinct manner by preventing clustering of RAS on the membrane surface (Spencer-Smith et al., “Inhibition of RAS Function Through Targeting an Allosteric Regulatory Site,” Nat Chem Biol. 13 (1): 62-8 (2017) (doi: 10.1038/nchembio.2231; PMID: 27820802). Because of this mode of action, NS1 can inhibit HRAS- and KRAS-mediated signaling regardless of the identity of oncogenic mutations. The crystal structure also revealed that the difference in amino acid residue at position 135, with Arg in KRAS and HRAS and Lys in NRAS, is the major determinant of the isoform selectivity of NS1.

It would be desirable, therefore, to develop a polypeptide binding molecule that binds specifically and selectively to NRAS over KRAS and HRAS, i.e., reversing the specificity profile of the monobody NS1, and acts to inhibit NRAS signaling implicated in various cancers.

The present invention is directed to overcoming these and other deficiencies in the art.

### SUMMARY

A first aspect relates to a Neuroblastoma-RAS (“NRAS”) binding polypeptide, the binding polypeptide including a fibronectin type III (FN3) domain having one or more modified loop sequences including a modified FG loop amino acid sequence, a modified BC loop amino acid sequence, a modified CD loop amino acid sequence, a modified DE loop amino acid sequence, or a combination thereof, and optionally one or more modified beta strand sequences, wherein said one or more modified loop sequences and said one or more optional modified beta strand sequences enable selectively binding to NRAS but not Kirsten RAS (“KRAS”) or Harvey RAS (“HRAS”).

A second aspect relates to an NRAS binding polypeptide conjugate, the conjugate including a first portion and a second portion. The first portion includes the NRAS binding polypeptide according to the first aspect. The second portion is coupled to the first portion, said is selected from the group an E3 ubiquitin ligase ligand or an E3 ligase subunit.

In certain embodiments according to the second aspect, the conjugate also includes a delivery vehicle linked to one or both of the first and second portions.

A third aspect relates to a polynucleotide encoding the NRAS binding polypeptide of according to the first aspect or the NRAS binding polypeptide conjugate according to the second aspect. Also encompassed by this aspect are vectors and host cells that include the polynucleotide.

A fourth aspect relates to a delivery vehicle including the polynucleotide or vector according to the third aspect.

A fifth aspect relates to a fusion construct including a first portion that includes a polynucleotide encoding the NRAS binding polypeptide according to the first aspect, and a second portion that is operatively coupled to the first portion and includes a polynucleotide encoding a E3 ligase subunit, or an imaging protein or peptide thereof.

A sixth aspect relates to a pharmaceutical composition. The pharmaceutical composition includes a pharmaceutically acceptable carrier and the NRAS binding polypeptide of according to the first aspect, the NRAS binding polypeptide conjugate according to the second aspect, the isolated polynucleotide or vector according to the third aspect, or the fusion construct according to the fifth aspect.

A seventh aspect relates to a method for treating cancer in a subject, the method including the step of administering, to the subject having cancer, the pharmaceutical composition of according to the sixth aspect in an amount effective to treat the cancer.

An eighth aspect relates to a method of screening agents for capability of binding NRAS. This method includes the steps of providing an affinity complex that includes NRAS and an NRAS binding polypeptide according to the first aspect that is non-covalently bound to NRAS; and exposing the complex to an agent and assessing whether the agent displaces the NRAS binding polypeptide from NRAS.

Because of their selectivity for NRAS over KRAS and HRAS, the NRAS-selective monobodies are expected to be inhibitors of NRAS-mediated signaling and tumorigenesis, and they are agnostic to the identity of NRAS mutations. Because the disclosed monobodies bind to a region of NRAS that is equivalent to the epitope of NS1 on HRAS and KRAS, the results presented in the accompanying Examples strongly support the expectation that these NRAS-selective monobodies, when introduced into cells as genetically encoded reagents or by other means, will be inhibitors of NRAS-mediated signaling and tumor growth. The ability to use one reagent to inhibit multiple NRAS mutations will be advantageous over an approach in which each mutation requires a separate reagent.

### BRIEF DESCRIPTION OF THE DRAWINGS

FIG. 1 is an alignment of NRAS-selective monobodies. The amino acid sequences of selected monobodies are shown beneath the design of the monobody library (consensus SEQ ID NO: 69). “X” in the library sequence denotes a mixture of 30% Tyr, 15% Ser, 10% Gly, 5% Phe, 5% Trp and 2.5% each of all the other amino acids except for Cys; “O” denotes a mixture of Asn, Asp, His, Ile, Leu, Phe, Tyr and Val; “U” denotes a mixture of His, Leu, Phe and Tyr; and “Z” denotes a mixture of Ala, Glu, Lys and Thr. Beneath the library is an alignment of the amino acid sequences of Mb(NRAS_9) (SEQ ID NO: 68), Mb(NRAS_19) (SEQ ID NO: 12), Mb(NRAS_20) (SEQ ID NO: 13), and Mb(NRAS_24) (SEQ ID NO: 14).

FIGS. 2A-C show biolayer interferometry (BLI) sensorgrams measuring the interactions of Mb(NRAS_19) (FIG. 2A), Mb(NRAS_20) (FIG. 2B) and Mb(NRAS_24) (FIG. 2C) with NRAS, HRAS and KRAS. The nucleotide state of RAS proteins is indicated. The dark gray lines (red in color version) in the NRAS panels show the global fitting of the 1:1 binding model. The deduced K_{D} values are also shown. The interactions of these monobodies with HRAS and KRAS were too weak to determine K_{D} values. In these experiments a biotinylated monobody was immobilized on streptavidin coated BLI tip, and then binding of RAS proteins was measured.

FIGS. 3A-3B show BLI sensorgrams measuring binding of Mb(NRAS_19) (SEQ ID NO: 12), Mb(NRAS_20) (SEQ ID NO: 13), and Mb(NRAS_24) (SEQ ID NO: 14) to NRAS (K135R) (FIG. 3A), and that of NS1 (FIG. 3B). FIG. 3C is a BLI sensorgram showing a competition binding assay between NS1 and NRAS-selective monobodies. In this experiment, biotinylated NS1 was immobilized on streptavidin coated BLI tip, and subsequently NRAS (K135R) was captured by NS1 (step 1). Subsequent addition of NRAS-selective monobodies generated negligible binding signals (steps 2-4).

FIGS. 4A-4B illustrate the sequence of Mb(NRAS_24) (SEQ ID NO: 14) and the sequences of the FG loop mutations, which when inserted into Mb(NRAS_24), the resulting monobody retains binding to NRAS. Mutated residues in each FG loop sequence are shown in bold typeface.

### DETAILED DESCRIPTION

The present invention relates generally to Neuroblastoma-RAS (“NRAS”) binding polypeptides, NRAS binding peptide conjugates, and polynucleotides encoding either the NRAS binding peptides or NRAS binding peptide conjugates. The disclosure also relates to methods of using these NRAS binding polypeptides, NRAS binding peptide conjugates, and polynucleotides encoding the same for the treatment of oncogenic NRAS cancers and related conditions.

### Definitions

Before the NRAS binding polypeptides, conjugates, polynucleotides, compositions and methods are described, it is to be understood that this invention is not limited to the particular polypeptides, conjugates, polynucleotides, compositions or methodologies described, as these may vary. It is also to be understood that the terminology used in the description is for the purpose of describing the particular versions or embodiments only, and is not intended to limit the scope of embodiments herein which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of embodiments herein, the preferred materials and methods are now described.

As used herein, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes +10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of components but not the exclusion of any other component or group of components and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” is intended to be inclusive rather than exclusive. For example, a condition A or B is satisfied by any one of the following: A is true (or present) and B is false (or not present), A is false (or not present) and B is true (or present), and both A and B are true (or present). As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited component or group of components, but that no additional component or group of components can be added to the specified method, structure, or composition.

As used herein, the term “consists essentially of,” or variations such as “consist essentially of” or “consisting essentially of,” as used throughout the specification and claims, indicate the inclusion of any recited component or group of components, and the optional inclusion of any recited component or group of components that do not materially change the basic or novel properties of the specified method, structure or composition.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, non-human primates, humans, etc., more preferably a human.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., NRAS binding polypeptides or polynucleotides encoding the same), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Examples of algorithms that are suitable for determining percent sequence identity and sequence similarity are the BLAST and BLAST 2.0 algorithms, which are described in Altschul et al., J. Mol. Biol. 215:403-410 (1990); and Altschul et al., Nucleic Acids Res. 25:3389-3402 (1997), each of which is hereby incorporated by reference in its entirety. Software for performing BLAST analyses is publicly available through the National Center for Biotechnology Information. In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993), which is hereby incorporated by reference in its entirety). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector,” refers to e.g., any number of nucleic acids into which a desired sequence can be inserted, e.g., be restriction and ligation, for transport between genetic environments or for expression in a host cell. Nucleic acid vectors can be DNA or RNA. Vectors include, but are not limited to, plasmids, phage, phagemids, bacterial genomes, virus genomes, self-amplifying RNA, replicons.

As used herein, the term “host cell” refers to a cell comprising a nucleic acid molecule of the invention. The “host cell” can be any type of cell, e.g., a primary cell, a cell in culture, or a cell from a cell line. In one embodiment, a “host cell” is a cell transfected or transduced with a nucleic acid molecule of the invention. In another embodiment, a “host cell” is a progeny or potential progeny of such a transfected or transduced cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed polypeptide can be within the cytoplasm of a host cell, secreted into the extracellular milieu such as the growth medium of a cell culture, or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component, or another therapeutic or diagnostic reagent as disclosed herein. Also included within the definition are, for example, polypeptides containing one or more analogs of an amino acid (including, for example, unnatural amino acids, etc.), as well as other modifications known in the art.

The polypeptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

The term “isolated” can refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated polypeptide refers to one that can be administered to a subject as an isolated polypeptide; in other words, the polypeptide may not simply be considered “isolated” if it is adhered to a column or embedded in a gel. Moreover, an “isolated nucleic acid fragment” or “isolated peptide” is a nucleic acid or protein fragment that is not naturally occurring as a fragment and/or is not typically in the functional state.

### NRAS Binding Polypeptides

Accordingly, a first aspect of the disclosure relates to a NRAS binding polypeptide. This NRAS binding polypeptide comprises a fibronectin type III (FN3) domain having one or more of a modified FG loop amino acid sequence, a modified BC loop amino acid sequence, a modified CD loop amino acid sequence, a modified DE loop amino acid sequence, or any combination of the aforementioned modified loop sequences, and optionally one or more modified beta strand amino acid sequences. The one or more modified loop sequences, optionally together with the one or more modified beta strand amino acid sequences, enable selective binding of the polypeptide to NRAS, including wild-type NRAS and oncogenic NRAS mutants, while showing negligible binding, if any, to related RAS isoforms KRAS and HRAS.

The FN3 domain is an evolutionary conserved protein domain that is about 100 amino acids in length and possesses a beta sandwich structure. The beta sandwich structure of human FN3 comprises seven beta-strands, referred to as strands A, B, C, D, E, F, G, with six connecting loops, referred to as loops AB, BC, CD, DE, EF, and FG that exhibit structural homology to immunoglobulin binding domains. Three of the six loops, i.e., loops DE, BC, and FG, correspond topologically to the complementarity determining regions of an antibody, i.e., CDR1, CDR2, and CDR3. The remaining three loops are surface exposed in a manner similar to antibody CDR3. In accordance with the present disclosure, one or more of the loop regions and, optionally, one or more beta strands of each FN3 domain of the binding molecule are modified to enable specific binding to NRAS protein.

As used herein “specifically binds” or “specific binding” refers to the ability of the FN3 containing binding molecule of the disclosure to bind to a predetermined antigen, i.e., NRAS with a dissociation constant (K_{D}) of about 1×10⁻⁶ M or less, for example about 1×10⁻⁷ M or less, about 1×10⁻⁸ M or less, about 1×10⁻⁹ M or less, about 1×10⁻¹⁰ M or less. Typically, the FN3 domain binds to NRAS with a K_{D} that is at least ten-fold less, such as at least 20-fold, 50-fold, 100-fold, 500-fold, or 1000-fold less, than its K_{D} for a nonspecific antigen (e.g., BSA or casein), and in particular wildtype NRAS, for example, as measured by biolayer interferometry using an Octet Instrument (Sartorius). The NRAS binding peptides described in the accompanying examples display low nanomolar to sub-nanomolar binding affinity, including measured K_{D} of less than 100 nM, less than 80 nM, less than 50 nM, less than 40 nM, less than 30 nM, less than 20 nM, less than 15 nM, or less than 10 nM (such as less than 9 nM, 8 nM, 7 nM, 6 nM, 5 nM, 4 nM, 3 nM, 2 nM, or 1 nM).

The modified FN3 domain of the binding molecule of the present disclosure can be a FN3 domain derived from any of the wide variety of animal, yeast, plant, and bacterial extracellular proteins containing these domains. In one embodiment, the FN3 domain is derived from a mammalian FN3 domain. Exemplary FN3 domains include, for example and without limitation, any one of the 15 different FN3 domains present in human tenascin C, or the 15 different FN3 domains present in human fibronectin (FN), for example, the 10th fibronectin type III domain. Exemplary FN3 domains also include non-natural synthetic FN3 domains, such as those described in U.S. Pat. Publ. No. 2010/0216708 to Jacobs et al., which is hereby incorporated by reference in its entirety. Individual FN3 domains are referred to by domain number and protein name, e.g., the 10th FN3 domain of fibronectin (10FN3).

In some embodiments, the FN3 domain of the binding molecule is derived from the 10th FN3 domain of fibronectin (10FN3). In some embodiments, the FN3 domain of the binding molecule is derived from the human 10FN3 domain. The human 10FN3 domain has the amino acid sequence of SEQ ID NO:1 as shown below. The locations of the BC (residues 24-30), CD (residues 40-45), DE (residues 51-55), EF (residues 60-66), and FG (residues 75-86) loops are shown in bold typeface within the wild-type sequence of SEQ ID NO: 1. Locations of other amino acid residues referenced in this disclosure are also identified within SEQ ID NO: 1 by their position.

(SEQ ID NO: 1)
VSDVPRDLEVVAATPTSLLISWDA₂₄PAVTVR₃₀Y₃₁YR₃₃ITYGE
TG₄₀GNSPV₄₅QE₄₇FT₄₉VP₅₁GSKS₅₅TATIS₆₀GLKPGV₆₆DYTIT
VYAV₇₅TGRGDSPASSK₈₆PISINYRT

In accordance with the present disclosure, one or more of the loop regions or selected residues within one or more of these loop regions are modified to enable NRAS binding specificity and affinity. Suitable modifications include amino acid residue substitutions, insertions, and/or deletions. In one aspect, amino acid residues in at least one, at least two, at least three, at least four, at least five, or all six of the loop regions are altered for NRAS binding specificity and affinity. In one embodiment, one or more amino acid modifications within the loop regions at or about residues 24-30 (BC loop), 40-45 (CD loop), 51-55 (DE loop), and/or 75-86 (FG loop) of SEQ ID NO: 1 form the NRAS binding region. In another embodiment, one or more amino acid modification within any one of these loop regions enable NRAS binding, including the FG loop only, the BC loop only, the CD loop only, the DE loop only, a combination of any two of those loop sequences (such as FG+BC, FG+CD, FG+DE, CD+BC, CD+DE, or BC+DE), or a combination of any three of those loop sequences, or all four of those loop sequences, optionally with the modification of one or more of the beta strands identified above.

In some embodiments, the NRAS binding molecule of the present disclosure comprises a modified BC loop. In some embodiments, the modified BC loop comprises the amino acid sequence of APAVTVX₇ (SEQ ID NO: 2), where X₇ is any amino acid residue, preferably Asp (D) or Glu (E). In some embodiments the modified BC loop is APAVTVD (SEQ ID NO: 3), or a BC loop having an amino acid sequence having about 85% sequence identity to SEQ ID NO: 3 (i.e., 6 of 7 residues identical).

In some embodiments, the NRAS binding molecule of the present disclosure comprises a modified CD loop. In some embodiments, the modified CD loop comprises the amino acid sequence of GX₂YX₄X₅X₆X₇ (SEQ ID NO: 4), where each of X₂, X₄, X₅, X₆, and X₇ is any amino acid residue. In certain embodiments, X₂ is Ser (S), Thr (T), Ala (A), or Gly (G); X₄ is Tyr (Y) or Ala (A); X₅ is Pro (P) or Trp (W); X₆ is Tyr (Y) or Ala (A); and X₇ is Tyr (Y) or Ala (A). In some embodiments the modified CD loop is GSYYPYA (SEQ ID NO: 5), GAYYPYA (SEQ ID NO: 6), GGYAWAY (SEQ ID NO: 7), or a CD loop having an amino acid sequence having ~85% sequence identity to one of SEQ ID NOS: 5-7 (i.e., 6 of 7 residues identical).

In some embodiments, the NRAS binding molecule of the present disclosure comprises a wildtype DE loop comprising the amino acid sequence of PGSKS (SEQ ID NO: 9). However, in other embodiments, the DE loop can be modified, having the amino acid sequence of X₁GSX4X₅, (SEQ ID NO: 8), where each of X₁, X₄, and X₅ is any amino acid residue, or otherwise. having an amino acid sequence having about 80% identity to SEQ ID NO: 9 (4 of 5 residues identical).

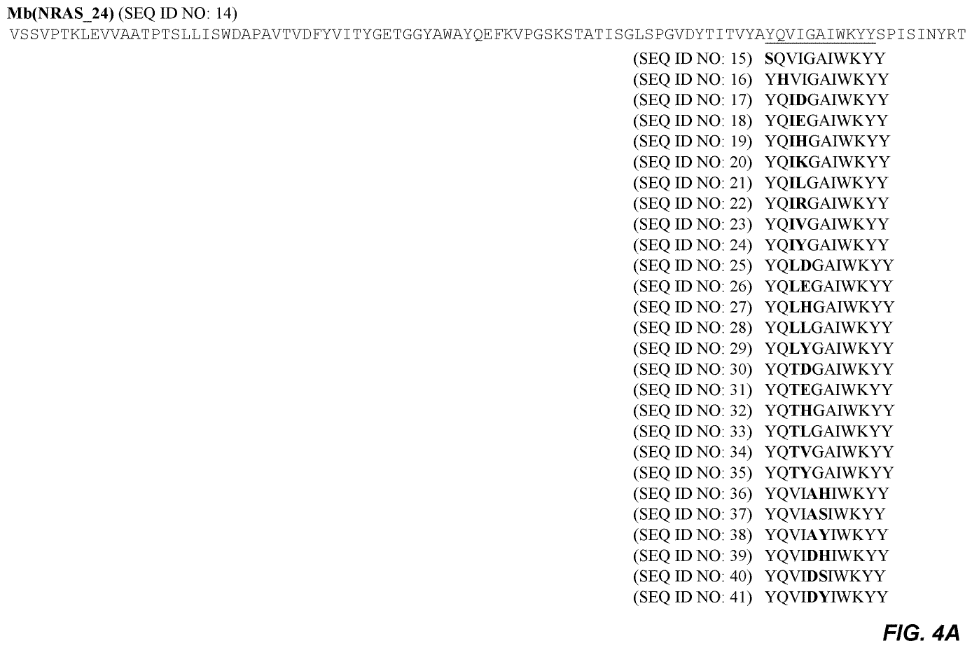

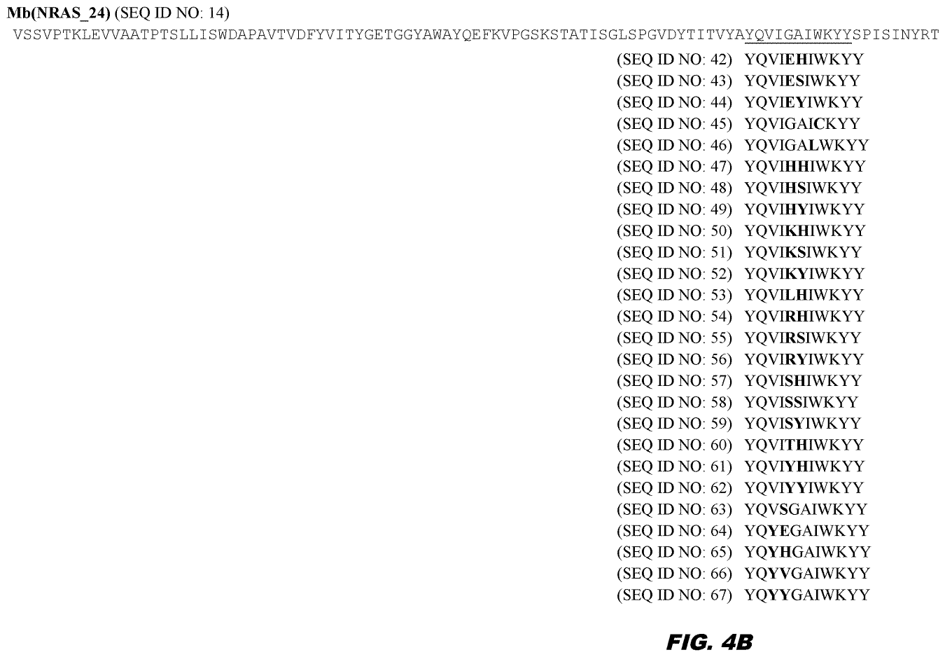

In some embodiments, the NRAS binding molecule of the present disclosure comprises a modified FG loop. In some embodiments, the modified FG loop comprises the amino acid sequence of (S/Y)-(Q/H)-X₃X₄X₅X₆-(I/L)-(W/C)-KYY (SEQ ID NO: 10) (see FIG. 4), where each of X₃, X₄, X₅, and X₆ is any amino acid residue, more preferably X₃ is Val (V), Tyr (Y), Thr (T), Leu (L), or Ile (I), X₄ is Ile (I), Asp (D), Glu (E), His (H), Leu (L), Lys (K), Arg (R), Val (V), Tyr (Y), or Ser(S), X₅ is Gly (G), Ala (A), Asp (D), Glu (E), His (H), Lys (K), Leu (L), Arg (R), Ser(S), Thr (T), or Tyr (Y), and X₆ is Ala (A), His (H), Ser(S), or Tyr (Y). In some embodiments, the modified FG loop is selected from any one of the modified FG loops of SEQ ID NOS: 11 and 15-67 (see Table 1 and FIG. 4), or an FG loop having an amino acid sequence having at least 50% sequence identity (i.e., 6 or more of 11 residues identical), at least 64% sequence identity (i.e., 7 or more of 11 residues identical), at least about 73% sequence identity (i.e., 8 or more of 11 residues identical), at least about 82% sequence identity (i.e., 9 or more of 11 residues identical), or at least about 91% sequence identity (i.e., 10 or more of 11 residues identical) to one of the modified FG loops of SEQ ID NOS: 12-64.

TABLE 1
NRAS Monobody Loop Sequences

| | | SEQ | | SEQ | | SEQ | | SEQ |
|---|---|---|---|---|---|---|---|---|
| | BC Loop | ID | CD Loop | ID | DE Loop | ID | FG Loop | ID |
| Name | Sequence | NO: | Sequence | NO: | Sequence | NO: | Sequence | NO: |
| NRAS_19 | APAVTVD | 3 | GSYYPYA | 5 | PGSKS | 9 | YQVIGAIWKYYS | 11 |
| NRAS_20 | APAVTVD | 3 | GAYYPYA | 6 | PGSKS | 9 | YQVIGAIWKYYS | 11 |
| NRAS_24 | APAVTVD | 3 | GGYAWAY | 7 | PGSKS | 9 | YQVIGAIWKYYS | 11 |
| Consensus | APAVTVD | 3 | GXYXXXX | 4 | PGSKS | 9 | YQVIGAIWKYYS | 11 |

As discussed above, FN3 domains contain two sets of CDR-like loops on the opposite faces of the molecule. The two sets of loops are separated by beta-strands (regions of the domain that are between the loops) that form the center of the FN3 structure. Like the loops, these beta-strands can be altered to enhance target molecule binding specificity and affinity. Preferably, some or all of the surface exposed residues in the beta strands are randomized without affecting (or minimally affecting) the inherent stability of the FN3 domain. In some embodiments, one or more of residues in one or more beta-strands is modified to enable interaction with NRAS. Suitable modifications include amino acid substitutions, insertions, and/or deletions. For example, one or more amino acid residues of the A beta strand, the B beta strand, the C beta strand, the D beta strand, the E beta strand, the F beta strand, or the G beta strand may be modified to enable NRAS binding or to enhance the specificity or affinity of NRAS binding.

In certain embodiments, one or more amino acid residues of the C and/or D beta-strands are modified for binding to the NRAS.

In some embodiments, the NRAS binding polypeptide described herein comprises one or more amino acid residue substitutions, additions, or deletions in the A beta strand or region upstream thereof.

In some embodiments, the NRAS binding polypeptide comprises an amino acid substitution at one or more resides corresponding to residues Y31 and R33 of SEQ ID NO: 1.

In some embodiments, the amino acid substitution is tyrosine to phenylalanine substitution at the amino acid residue corresponding to the tyrosine at position 31 (Y31F), tyrosine to histidine substitution at the amino acid residue corresponding to the tyrosine at position 31 (Y31H), tyrosine to lysine substitution at the amino acid residue corresponding to the tyrosine at position 31 (Y31K), or tyrosine to leucine substitution at the amino acid residue corresponding to the tyrosine at position 31 (Y31L) of SEQ ID NO: 1.

In some embodiments, the amino acid substitution is arginine to valine substitution at the amino acid residue corresponding to the arginine at position 33 (R33V), arginine to aspartic acid substitution at the amino acid residue corresponding to the arginine at position 33 (R33D), arginine to alanine substitution at the amino acid residue corresponding to the arginine at position 33 (R33A), arginine to cytosine substitution at the amino acid residue corresponding to the arginine at position 33 (R33C), arginine to glutamic acid substitution at the amino acid residue corresponding to the arginine at position 33 (R33E), arginine to glycine substitution at the amino acid residue corresponding to the arginine at position 33 (R33G), arginine to histidine substitution at the amino acid residue corresponding to the arginine at position 33 (R33H), arginine to isoleucine substitution at the amino acid residue corresponding to the arginine at position 33 (R33I), arginine to lysine substitution at the amino acid residue corresponding to the arginine at position 33 (R33K), arginine to leucine substitution at the amino acid residue corresponding to the arginine at position 33 (R33L), arginine to methionine substitution at the amino acid residue corresponding to the arginine at position 33 (R33M), arginine to asparagine substitution at the amino acid residue corresponding to the arginine at position 33 (R33N), arginine to glutamine substitution at the amino acid residue corresponding to the arginine at position 33 (R33Q), arginine to serine substitution at the amino acid residue corresponding to the arginine at position 33 (R33S), arginine to threonine substitution at the amino acid residue corresponding to the arginine at position 33 (R33T), arginine to tryptophan substitution at the amino acid residue corresponding to the arginine at position 33 (R33W), or arginine to tyrosine substitution at the amino acid residue corresponding to the arginine at position 33 (R33Y) of SEQ ID NO: 1.

In some embodiments, the NRAS binding polypeptide comprises an amino acid substitution at one or more residues corresponding to residue E47 or R49 of SEQ ID NO: 1.

In some embodiments, the amino acid substitution is glutamic acid to alanine substitution at the amino acid residue corresponding to the glutamic acid at position 47 (E47A), glutamic acid to cysteine substitution at the amino acid residue corresponding to the glutamic acid at position 47 (E47C), glutamic acid to aspartic acid substitution at the amino acid residue corresponding to the glutamic acid at position 47 (E47D), glutamic acid to phenylalanine substitution at the amino acid residue corresponding to the glutamic acid at position 47 (E47F), glutamic acid to glycine substitution at the amino acid residue corresponding to the glutamic acid at position 47 (E47G), glutamic acid to histidine substitution at the amino acid residue corresponding to the glutamic acid at position 47 (E47H), glutamic acid to isoleucine substitution at the amino acid residue corresponding to the glutamic acid at position 47 (E471), glutamic acid to lysine substitution at the amino acid residue corresponding to the glutamic acid at position 47 (E47K), glutamic acid to leucine substitution at the amino acid residue corresponding to the glutamic acid at position 47 (E47L), glutamic acid to methionine substitution at the amino acid residue corresponding to the glutamic acid at position 47 (E47M), glutamic acid to asparagine substitution at the amino acid residue corresponding to the glutamic acid at position 47 (E47N), glutamic acid to proline substitution at the amino acid residue corresponding to the glutamic acid at position 47 (E47P), glutamic acid to glutamine substitution at the amino acid residue corresponding to the glutamic acid at position 47 (E47Q), glutamic acid to arginine substitution at the amino acid residue corresponding to the glutamic acid at position 47 (E47R), glutamic acid to serine substitution at the amino acid residue corresponding to the glutamic acid at position 47 (E47S), glutamic acid to threonine substitution at the amino acid residue corresponding to the glutamic acid at position 47 (E47T), glutamic acid to valine substitution at the amino acid residue corresponding to the glutamic acid at position 47 (E47V), glutamic acid to tryptophan substitution at the amino acid residue corresponding to the glutamic acid at position 47 (E47W), or glutamic acid to tyrosine substitution at the amino acid residue corresponding to the glutamic acid at position 47 (E47Y) of SEQ ID NO: 1.

In some embodiments, the amino acid substitution is threonine to lysine substitution at the amino acid residue corresponding to the threonine at position 49 (T49K), threonine to alanine substitution at the amino acid residue corresponding to the threonine at position 49 (T49A), threonine to histidine substitution at the amino acid residue corresponding to the threonine at position 49 (T49H), threonine to isoleucine substitution at the amino acid residue corresponding to the threonine at position 49 (T491), threonine to proline substitution at the amino acid residue corresponding to the threonine at position 49 (T49P), threonine to glutamine substitution at the amino acid residue corresponding to the threonine at position 49 (T49Q), threonine to arginine substitution at the amino acid residue corresponding to the threonine at position 49 (T49R), threonine to serine substitution at the amino acid residue corresponding to the threonine at position 49 (T49S), or threonine to valine substitution at the amino acid residue corresponding to the threonine at position 49 (T49V) of SEQ ID NO: 1.

A number of other beta strand substitutions are identified in EP2598529A2 to Loew et al., which is hereby incorporated by reference in its entirety. Those substitutions can be incorporated in the NRAS binding peptides of the present invention.

In some embodiments, the FN3 domain comprises an amino acid sequence that is at least 80% identical to an amino acid sequence of SEQ ID NO: 12. In some embodiments, the FN3 domain comprises an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to an amino acid sequence of SEQ ID NO: 12. In some embodiments, the FN3 domain comprises an amino acid sequence of SEQ ID NO: 12.

Mb(NRAS_19)
(SEQ ID NO: 12)
VSSVPTKLEVVAATPTSLLISWDAPAVTVDFYVITYGETGSYY
PYAQEFKVPGSKSTATISGLSPGVDYTITVYAYQVIGAIWKYY
SPISINYRT

In SEQ ID NO: 12, bold/italicized residues are modified from the wildtype 10FN3 domain of SEQ ID NO: 1. In one embodiment, one or more amino acid residues of the A, B, C, D, E, and/or F beta-strands are modified in SEQ ID NO: 13 for binding to NRAS. For example, the A strand includes D3S, R6T, and D7K substitutions, the C strand includes Y31F and R33V substitutions. In addition, BC loop includes a R30D substitution, and the EF loop includes a K63S substitution.

In some embodiments, the FN3 domain comprises an amino acid sequence that is at least 80% identical to an amino acid sequence of SEQ ID NO: 13. In some embodiments, the FN3 domain comprises an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to an amino acid sequence of SEQ ID NO: 13. In some embodiments, the FN3 domain comprises an amino acid sequence of SEQ ID NO: 13.

Mb(NRAS_20)
(SEQ ID NO: 13)
VSSVPTKLEVVAATPTSLLISWDAPAVTVDFYVITYGETGAYY
PYAQEFKVPGSKSTATISGLSPGVDYTITVYAYQVIGAIWKYY
SPISINYRT

In SEQ ID NO: 13, bold/italicized residues are modified from the wildtype 10FN3 domain of SEQ ID NO: 1. In one embodiment, one or more amino acid residues of the A, B, C, D, E, and/or F beta-strands are modified in SEQ ID NO: 13 for binding to NRAS. For example, the A strand includes D3S, R6T, and D7K substitutions, and the C strand includes Y31F and R33V substitutions. In addition, BC loop includes a R30D substitution, and the EF loop includes a K63S substitution.

In some embodiments, the FN3 domain comprises an amino acid sequence that is at least 80% identical to an amino acid sequence of SEQ ID NO: 14. In some embodiments, the FN3 domain comprises an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to an amino acid sequence of SEQ ID NO: 14. In some embodiments, the FN3 domain comprises an amino acid sequence of SEQ ID NO: 14.

Mb(NRAS_24)
(SEQ ID NO: 14)
VSSVPTKLEVVAATPTSLLISWDAPAVTVDFYVITYGETGGYA
WAYQEFKVPGSKSTATISGLSPGVDYTITVYAYQVIGAIWKYY
SPISINYRT

In SEQ ID NO: 14, bold/italicized residues are modified from the wildtype 10FN3 domain of SEQ ID NO: 1. In one embodiment, one or more amino acid residues of the A, B, C, D, E, and/or F beta-strands are modified in SEQ ID NO: 14 for binding to NRAS. For example, the A strand includes D3S, R6T, and D7K substitutions, and the C strand includes Y31F and R33V substitutions. In addition, BC loop includes a R30D substitution, and the EF loop includes a K63S substitution.

In some embodiments, the FN3 domain comprises an amino acid sequence that is at least 80% identical to an amino acid sequence of SEQ ID NO: 68. In some embodiments, the FN3 domain comprises an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to an amino acid sequence of SEQ ID NO: 68. In some embodiments, the FN3 domain comprises an amino acid sequence of SEQ ID NO: 68.

Mb(NRAS_9)
(SEQ ID NO: 68)
VSSVPTKLEVVAATPTSLLISWDAPAVTVDFYVITYGETGSYY
PYAQEFKVPGSKSTATISGLKPGVDYTITVYAYQVIGAIWKYY
SPISINYRT

In the above-identified monobody of Mb(NRAS_9), the A, C and D beta strands contain the same amino acid substitutions noted above for the other monobody sequences. In addition, the EF loop comprises a wildtype K63 residue rather than the K63S substitution that is presented in the EF loops of Mb(NRAS_19), Mb(NRAS_20), and Mb(NRAS_24). The K63S substitution has previously been shown to have a minimal effect on binding (Wojcik et al., “A Potent and Highly Specific FN3 Monobody Inhibitor of the Abl SH2 Domain,” Nat Struct Mol Biol 17 (4): 519-27 (PMID 2926940) (2010), which is hereby incorporated by reference in its entirety).

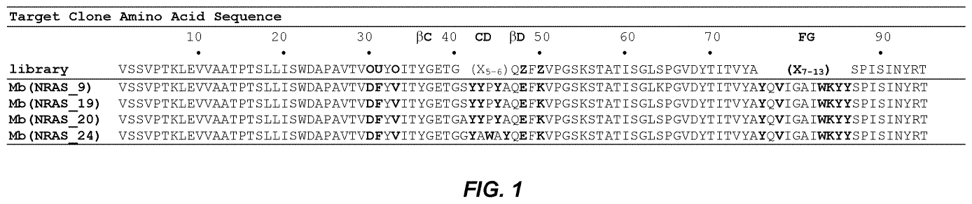

An alignment of the four NRAS binding monobodies is presented in FIG. 1.

Further modifications of these amino acid sequences are also contemplated, including replacing the FG loop sequence in any one of the above identified monobodies with an FG loop sequence according to one of SEQ ID NOS: 15-67 illustrated in FIGS. 4A-4B, or replacing an FG loop sequence with any two, three, four, five, or six mutations illustrated in the FG loop sequences of FIGS. 4A-4B.

The NRAS binding polypeptides as disclosed here can be prepared according to any suitable procedure.

According to one approach, the NRAS binding polypeptide of the present invention can be synthesized by standard peptide synthesis operations. These include both FMOC (9-fluorenylmethyloxy-carbonyl) and tBoc (tert-butyloxy-carbonyl) synthesis protocols that can be carried out on automated solid phase peptide synthesis instruments including, without limitation, the Applied Biosystems 431A, 433A synthesizers, Peptide Technologies Symphony, or large-scale Sonata or CEM Liberty automated solid phase peptide synthesizers. The use of alternative peptide synthesis instruments is also contemplated. Peptides prepared using solid phase synthesis are recovered in a substantially pure form.

Alternatively, as discussed below, the NRAS binding polypeptide can be recombinantly produced using recombinant molecular techniques to generate host cells that contain and express a transgene that results in the production of the NRAS binding polypeptide by the recombinant host cell. Upon growing the host cells under sufficient conditions to express the NRAS binding polypeptide, the recombinantly produced NRAS binding polypeptide can be recovered and then purified using standard techniques such as high-performance liquid chromatography, affinity chromatography, and/or size-exclusion chromatography. Other known techniques can be used alone or in combination with these chromatography techniques.

### NRAS Binding Polypeptide Conjugates

Another aspect of the present disclosure relates to a NRAS binding peptide conjugate comprising a first portion and a second portion. The first portion of the conjugate comprises the NRAS binding polypeptide as described supra. The second portion of the conjugate, which is coupled to the first portion of the conjugate, is selected from a diagnostic moiety, an E3 ligase subunit or an E3 ubiquitin ligase binding ligand.

In accordance with this aspect of the present disclosure, the first and second portions of the conjugate are covalently coupled to each other directly or via a linker. The first and second portions may be directly fused and generated by standard cloning and expression techniques. Alternatively, well known chemical coupling methods may be used to attach the portions directly or via a peptide or other linker to produce NRAS binding peptide conjugates as described herein. For example, covalent conjugation of the first and second portions can be accomplished via lysine side chains using an activated ester or isothiocyanate, or via cysteine side chains with a maleimide, haloacetyl derivative or activated disulfide. Site specific conjugation of the first and second portions can also be accomplished by incorporating unnatural amino acids, self-labeling tags (e.g., SNAP or DHFR), or a tag that is recognized and modified specifically by another enzyme such as sortase A, lipoic acid ligase, and formylglycine-generating enzyme. In some embodiments, site specific conjugation of the first and second portions is achieved by the introduction of cysteine residue either at the C-terminus of the NRAS binding molecule or at a specific site as described by Goldberg et al., “Engineering a Targeted Delivery Platform Using Centyrins,” Protein Engineering, Design & Selection 29 (12): 563-572 (2016), which is hereby incorporated by reference in its entirety.

In some embodiments, the first and second portions of the NRAS binding peptide conjugate are coupled together via a linker. In some embodiments, the linker is an amino acid linker. In some embodiments, the amino acid linker is a cleavable linker. In some embodiments, the amino acid linker is a non-cleavable linker. Suitable linkers include peptides composed of repetitive modules of one or more of the amino acids, such as glycine and serine or alanine and proline. Exemplary linker peptides include, e.g., (Gly-Gly) n, (Gly-Ser) n, (Gly3-Ser) n, (Ala-Pro) n wherein n is an integer from 1-25. The length of the linker may be appropriately adjusted as long as it does not affect the function of the non-binding protein-drug conjugate. The standard 15 amino acid (Gly4-Ser) 3 linker peptide has been well-characterized and has been shown to adopt an unstructured, flexible conformation. In addition, this linker peptide does not interfere with assembly and activity of the domains it connects (Freund et al., “Characterization of the Linker Peptide of the Single-Chain Fv Fragment of an Antibody by NMR Spectroscopy,” FEBS 320:97 (1993), the disclosure of which is hereby incorporated by reference in its entirety).

In some embodiments, the second portion of the NRAS binding peptide conjugate is a E3 ubiquitin ligase subunit. Exemplary E3 ubiquitin ligase subunits include, without limitation, those selected from the group of von Hippel-Lindau (VHL); cereblon, XIAP, E3A; MDM2; Anaphase-promoting complex (APC); UBR5 (EDD1); SOCS/BC-box/eloBC/CUL5/RING; LNXp80; CBX4; CBLL1; HACE1; HECTD1; HECTD2; HECTD3; HECW1; HECW2; HERC1; HERC2; HERC3; HERC4; HUWE1; ITCH; NEDD4; NEDD4L; PPIL2; PRPF19; PIAS1; PIAS2; PIAS3; PIAS4; RANBP2; RNF4; RBX1; SMURF1; SMURF2; STUB1; TOPOR5; TRIP12; UBE3A; UBE3B; UBE3C; UBE4A; UBE4B; UBOX5; UBR5; WWP1; WWP2; Parkin; A20/TNFAIP3; AMFR/gp78; ARA54; beta-TrCP1/BTRC; BRCA1; CBL; CHIP/STUB1; E6; E6AP/UBE3A; F-box protein 15/FBXO15; FBXW7/Cdc4; GRAII/RNF128; HOIP/RNF31; cIAP-1/HIAP-2; cIAP-2/HIAP-1; cIAP (pan); ITCH/AIP4; KAP1; MARCH8; Mind Bomb 1/MIB1; Mind Bomb 2/MIB2; MuRF1/TRIM63; NDFIP1; NEDD4; NleL; Parkin; RNF2; RNF4; RNF8; RNF168; RNF43; SART1; Skp2; SMURF2; TRAF-1; TRAF-2; TRAF-3; TRAF-4; TRAF-5; TRAF-6; TRIM5; TRIM21; TRIM32; UBR5; and ZNRF3.

In another embodiment, the second portion of the NRAS binding peptide conjugate is E3 ubiquitin ligase ligands, which can be a small molecule ligand or peptide ligand.

Exemplary E3 ubiquitin ligase ligands include, without limitation, small molecule ligands such as thalidomide, lenalidomide, pomalidomide and analogs thereof known to bind to cereblon; cereblon ligands disclosed in US2016/0058872 and US2015/0291562, which are hereby incorporated by reference in their entirety; E3 ligase ligands that bind MDM2 include nutlin compounds such as nutlin 3a and nutlin 3, as well as those disclosed in any one of WO2012/121361; WO2014/038606; WO2010/082612; WO2014/044401; WO2009/151069; WO2008/072655; WO2014/100065; WO2014/100071; WO2014/123882; WO2014/120748; WO2013/096150; WO2015/161032; WO2012/155066; WO2012/065022; WO2011/060049; WO2008/036168; WO2006/091646; WO2012/155066; WO2012/065022; WO2011/153509; WO2013/049250; WO2014/151863; WO2014/130470; WO2014/134207; WO2014/200937; WO2015/070224; WO2015/158648; WO2014/082889; WO2013/178570; WO2013/135648; WO2012/116989; WO2012/076513; WO2012/038307; WO2012/034954; WO2012/022707; WO2012/007409; WO2011/134925; WO2011/098398; WO2011/101297; WO2011/067185; WO2011/061139; WO2011/045257; WO2010/121995; WO2010/091979; WO2010/094622; WO2010/084097; WO2009/115425; WO2009/080488; WO2009/077357; WO2009/047161A1; WO2008/141975A1, all of which are hereby incorporated by reference in their entirety; E3 ligase ligands that bind IAP include compounds disclosed in U.S. Pat. No. 9,096,544; WO 2015187998; WO 2015071393; U.S. Pat. Nos. 9,278,978; 9,249,151; US20160024055; US 20150307499; US20140135270; US20150284427; US20150259359; US20150266879; US 20150246882; US20150252072; US20150225449; U.S. Pat. No. 8,883,771, which are hereby incorporated by reference in their entirety; and E3 ligase ligands that bind VHL such as hydroxyproline compounds disclosed in WO2013/106643, and other compounds described in US2016/0045607, WO2014187777, US20140356322, and U.S. Pat. No. 9,249,153, Yang et al., “Discovery of thalidomide-based PROTAC small molecules as the highly efficient SHP2 degraders,” Eur J Med Chem. 218:113341 (2021), Crew et al., “Identification and Characterization of Von Hippel-Lindau-recruiting proteolysis targeting chimeras (PROTACs) of TANK-binding kinase 1,” J Med Chem. 61 (2): 583-98 (2018); which are hereby incorporated by reference in their entirety. This listing above is by no means exhaustive, and it is contemplated that other E3 ligase ligands can be used as the second portion of the conjugate.

In another embodiment, the second portion of the NRAS binding peptide conjugate comprises a second polypeptide. In some embodiments, the second polypeptide is a non-binding molecule. In some embodiments, the polypeptide is a second binding molecule. In some embodiments, the second binding molecule is an antibody or antibody binding domain thereof, as well as other synthetic binding proteins such as monobodies and DARPins.

As used herein, an antibody includes any protein or peptide containing molecule that comprises at least a portion of an immunoglobulin molecule, such as but not limited to, at least one, at least two, or at least three complementarity determining region (CDR) of a heavy or light chain, a heavy chain or light chain variable region, a heavy chain or light chain constant region, a framework region, or any portion thereof. Antibodies encompass full antibodies, digestion fragments, specified portions and variants thereof, including, without limitation, portions of antibodies that mimic the structure and/or function of an antibody or specified fragment or portion thereof, including, without limitation, single chain antibodies, single domain antibodies (i.e., antibody fragments comprising merely one variable domain, which might be VHH, VH or VL, that specifically bind an antigen or epitope independently of other V regions or domains). Functional fragments include antigen-binding fragments that bind to a particular target. For example, antibody fragments capable of binding to a particular target or portions thereof, include, but are not limited to, Fab (e.g., by papain digestion), Fab′ (e.g., by pepsin digestion and partial reduction) and F(ab′)2 (e.g., by pepsin digestion), Fd (e.g., by pepsin digestion, partial reduction and reaggregation), Fv or scFv (e.g., by molecular biology techniques) fragments.

In any embodiment, the pharmaceutically active moiety of the NRAS binding peptide conjugate is coupled to or packaged within a delivery vehicle. In certain embodiments, the delivery vehicle is a third portion of the conjugate, which is linked in the manner described above to the first and/or second portions of the conjugate. Accordingly, in some embodiments, the NRAS binding peptide conjugate comprises the NRAS binding polypeptide coupled to a delivery vehicle. In any embodiment, the delivery vehicle contains a pharmaceutically active moiety.

In accordance with this aspect of the disclosure, any suitable drug delivery vehicle known in the art can be coupled to the NRAS binding polypeptide to form the NRAS binding peptide conjugate as described herein. By way of example, the drug delivery vehicle can be a peptide or polypeptide responsible for cell uptake, a nanoparticle delivery vehicle, a polymer-based particle, or a lipid-based particle delivery vehicle known in the art (see, e.g., Xiao et al., “Engineering Nanoparticles for Targeted Delivery of Nucleic Acid Therapeutics in Tumor,” Mol. Ther. Meth. Clin. Dev. 12:1-18 (2019) and Ni et al., “Synthetic Approaches for Nucleic Acid Delivery: Choosing the Right Carriers,” Life 9 (3): 59 (2019), which are hereby incorporated by reference in their entirety), can be employed in the methods as described herein.

In alternative embodiments, the drug delivery vehicle is not linked per se to the conjugate, but the conjugate is encapsulated by, packaged within, or otherwise associated with the drug delivery vehicle. This is applicable to a number of the delivery vehicles identified above, including a nanoparticle delivery vehicle, a polymer-based particle, or a lipid-based particle.

Exemplary peptides or polypeptides suitable for cell uptake include, without limitation, an antibody or binding fragment thereof that binds a cell surface molecule, a cell penetrating peptide, or a toxin that facilitates cell uptake.

Suitable antibodies, or binding fragments thereof, that bind a cell surface molecule include, without limitation, those targeting a cell surface marker that is specific for a particular type of cell. Any number of cell specific surface markers are known in the art. In one embodiment, the cell specific surface marker is a tumor-specific antigen or cancer cell specific antigen, such as a cell surface protein expressed on oncogenic RAS cancer cells. Exemplary cancer cell specific surface markers (that the antibody, or binding fragment thereof, recognizes and specifically binds to) are selected from CUB domain-containing protein 1 (CDCP1), Intercellular adhesion molecule 1 (ICAM1), Integrin beta-5 (ITGB5), (5′-nucleotidase) NT5E, Tumor necrosis factor receptor superfamily member 3 (LTBR), Complement decay-accelerating factor (CD55), Aminopeptidase N (ANPEP), CD79, Trophoblast glycoprotein (TPBG), Integrin beta-1 (ITGB1), Prostaglandin F2 receptor negative regulator (PTGFRN), Integrin alpha-5 (ITGA5), and Exosome complex protein LRP1 (LRP1) (see e.g., Martinko et al., “Targeting RAS-driven Human Cancer Cells with Antibodies to Upregulated and Essential Cell-Surface Proteins, eLIFE 7: e31098 (2018), which is hereby incorporated by reference in its entirety). Other cancer cell specific antigen can also be used for targeting by the antibody or binding fragment thereof.

Suitable cell penetrating peptides include, without limitation, TAT, cR10, Pep-1, penetratin, and transportan. Other cell penetrating peptides can also be used.

Suitable toxins that facilitate cell uptake are typically bacterial, fungal, or viral toxin, and in certain embodiments these toxins may be attenuated so that their toxic effects are diminished or abolished. Exemplary attenuated toxins include, without limitation, attenuated anthrax toxin, an attenuated diphtheria toxin, and attenuated Shiga or Shiga-like toxin. Other toxins, particularly attenuated toxins, can also be used.

Alternatively, any of a variety of non-proteinaceous delivery vehicles can be used, including a nanoparticle delivery vehicle, a polymer-based particle, or a lipid-based particle delivery vehicle.

Suitable nanoparticle delivery vehicles comprise, without limitation, gold nanoparticles, calcium phosphate nanoparticles, cadmium (quantum dots) nanoparticles, iron oxide nanoparticles, as well as particles derived from any other solid inorganic materials as known in the art.

Suitable polymer-based particles or polyplex carriers comprise cationic polymers such as polyethylenimine (PEI), and/or cationic polymers conjugated to neutral polymers, like polyethylene glycol (PEG) and cyclodextrin. Other suitable PEI conjugates to facilitate nucleic acid molecule or expression vector delivery in accordance with the methods described herein include, without limitation, PEI-salicylamide conjugates and PEI-steric acid conjugate. Other synthetic cationic polymers suitable for use as a delivery vehicle material include, without limitation, poly-L-lysine (PLL), polyacrylic acid (PAA), polyamideamine-epichlorohydrin (PAE) and poly[2-(dimethylamino)ethyl methacrylate] (PDMAEMA). Natural cationic polymers suitable for use as delivery vehicle material include, without limitation, chitosan, poly(lactic-co-glycolic acid) (PLGA), gelatin, dextran, cellulose, and cyclodextrin.

Suitable lipid-based vehicles include cationic lipid based lipoplexes (e.g., 1,2-dioleoyl-3trimethylammonium-propane (DOTAP)), neutral lipids based lipoplexes (e.g., cholesterol and dioleoylphosphatidyl ethanolamine (DOPE)), anionic lipid based lipoplexes (e.g., cholesteryl hemisuccinate (CHEMS)), and pH-sensitive lipid lipoplexes (e.g., 2,3-dioleyloxy-N-[2 (sperminecarboxamido)ethyl]-N,N-dimethyl-1-propanaminium trifluoroacetate (DOSPA)). Other suitable lipid-based delivery particles incorporate ionizable DOSPA in lipofectamine and DLin-MC3-DMA ((6Z,9Z,28Z,31Z)-heptatriaconta-6,9,28,31-tetraen-19-yl-4-(dimethylamino) butanoate).

### Polynucleotides, Vectors, and Host Cells

Another aspect of the present disclosure is directed to polynucleotides encoding the NRAS binding molecules or the NRAS binding peptide conjugates described herein. The nucleic acid molecules of the present disclosure include isolated polynucleotides, portions of expression vectors or portions of linear DNA sequences, including linear DNA sequences used for in vitro transcription/translation, vectors compatible with prokaryotic, eukaryotic or filamentous phage expression, secretion and/or display of the compositions or directed mutagens thereof.

In one embodiment isolated polynucleotides of the present disclosure include those encoding the binding molecules described supra. Exemplary isolated polynucleotide molecules include those encoding a FN3 domain comprising one or more of a BC loop amino acid sequence of SEQ ID NO: 3, an CD loop amino acid sequence of SEQ ID NO: 5, a DE loop amino acid sequence of SEQ ID NO: 9, and an FG loop amino acid sequence of SEQ ID NO: 11. In some embodiments, the FN domain encoded by the polynucleotide further comprises an amino acid substitution in one or more beta strands as discussed supra, including at one or more residues corresponding to residues Y31, R33, and/or T49 of SEQ ID NO: 1. In some embodiments, the FN3 domain encoded by the polynucleotide of the disclosure comprises an amino acid sequence that is at least 80% identical to an amino acid sequence of SEQ ID NO: 12. In some embodiments, the FN3 domain encoded by the polynucleotide of the present disclosure comprises an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to an amino acid sequence of SEQ ID NO: 12. In some embodiments, the polynucleotide of the present disclosure encodes an FN3 domain comprising an amino acid sequence of SEQ ID NO: 12 (Mb(NRAS_19)).

In one embodiment, the exemplary isolated polynucleotide encoding the FN3 domain of SEQ ID NO: 12 (Mb(NRAS_19)) is a DNA molecule that is codon-optimized for expression in human cells. One such polynucleotide includes the DNA sequence according to SEQ ID NO: 70 as follows:

GTGTCCAGCGTGCCCACCAAGCTGGAAGTGGTCGCCGCTACAC
CTACAAGCCTGCTGATCTCTTGGGACGCCCCTGCTGTGACCGT
GGACTTCTACGTGATCACCTACGGCGAGACAGGCAGCTACTAT
CCTTACGCCCAGGAGTTCAAGGTGCCAGGATCTAAAAGCACCG
CCACCATCTCCGGCCTGAGCCCTGGCGTGGATTACACCATTAC
AGTGTACGCCTACCAGGTGATCGGCGCCATCTGGAAGTACTAC
AGCCCCATCAGCATCAACTACAGAACCTAA

This polynucleotide can be coupled to any of a variety of promoter and enhancer sequences, as well as 3′ polyadenylation sequences, which are operable in human cells.

In one embodiment isolated polynucleotides of the present disclosure include those encoding the binding molecules described supra. Exemplary isolated polynucleotide molecules include those encoding a FN3 domain comprising one or more of a BC loop amino acid sequence of SEQ ID NO: 3, an CD loop amino acid sequence of SEQ ID NO: 6, a DE loop amino acid sequence of SEQ ID NO: 9, and an FG loop amino acid sequence of SEQ ID NO: 11. In some embodiments, the FN domain encoded by the polynucleotide further comprises an amino acid substitution in one or more beta strands as discussed supra, including at one or more residues corresponding to residues Y31, R33, and/or T49 of SEQ ID NO: 1. In some embodiments, the FN3 domain encoded by the polynucleotide of the disclosure comprises an amino acid sequence that is at least 80% identical to an amino acid sequence of SEQ ID NO: 13. In some embodiments, the FN3 domain encoded by the polynucleotide of the present disclosure comprises an amino acid sequence that at least 90% identical, at least 91% identical, at least 92% identical, at least 93% identical, at least 94% identical, at least 95% identical, at least 96% identical, at least 97% identical, at least 98% identical, or at least 99% identical to an amino acid sequence of SEQ ID NO: 13. In some embodiments, the polynucleotide of the present disclosure encodes an FN3 domain comprising an amino acid sequence of SEQ ID NO: 13 (Mb(NRAS_20)).

In one embodiment, the exemplary isolated polynucleotide encoding the FN3 domain of SEQ ID NO: 13 (Mb(NRAS_20)) is a DNA molecule that is codon-optimized for expression in human cells. One such polynucleotide includes the DNA sequence according to SEQ ID NO: 71 as follows:

GTTTCTAGCGTGCCTACAAAGCTGGAAGTGGTCGCCGCCACCC
CTACCAGCCTGCTGATCAGCTGGGACGCCCCTGCCGTGACCGT
GGACTTCTACGTGATCACCTACGGCGAGACAGGCGCTTATTAC
CCCTACGCCCAGGAGTTCAAGGTGCCAGGCAGCAAGTCCACCG
CCACAATCTCCGGCCTGAGCCCTGGCGTGGATTACACCATCAC
AGTGTACGCCTACCAGGTGATTGGAGCTATCTGGAAATACTAC
AGCCCCATCTCTATCAACTACAGAACCTAA

This polynucleotide can be coupled to any of a variety of promoter and enhancer sequences, as well as 3′ polyadenylation sequences, which are operable in human cells.

In one embodiment isolated polynucleotides of the present disclosure include those encoding the binding molecules described supra. Exemplary isolated polynucleotide molecules include those encoding a FN3 domain comprising one or more of a BC loop amino acid sequence of SEQ ID NO: 3, an CD loop amino acid sequence of SEQ ID NO: 7, a DE loop amino acid sequence of SEQ ID NO: 9, and an FG loop amino acid sequence of SEQ ID NO: 11. In some embodiments, the FN domain encoded by the polynucleotide further comprises an amino acid substitution in one or more beta strands as discussed supra, including at one or more residues corresponding to residues Y31, R33, and/or T49 of SEQ ID NO: 1. In some embodiments, the FN3 domain encoded by the polynucleotide of the disclosure comprises an amino acid sequence that is at least 80% identical to an amino acid sequence of SEQ ID NO: 14. In some embodiments, the FN3 domain encoded by the polynucleotide of the present disclosure comprises an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to an amino acid sequence of SEQ ID NO: 14. In some embodiments, the polynucleotide of the present disclosure encodes an FN3 domain comprising an amino acid sequence of SEQ ID NO: 14 (Mb(NRAS_24)).

In one embodiment, the exemplary isolated polynucleotide encoding the FN3 domain of SEQ ID NO: 14 (Mb(NRAS_24)) is a DNA molecule that is codon-optimized for expression in human cells. One such polynucleotide includes the DNA sequence according to SEQ ID NO: 72 as follows:

GTGTCCAGCGTGCCCACAAAGCTGGAAGTGGTTGCCGCTACAC
CTACCAGCCTGCTGATCTCTTGGGACGCCCCTGCCGTGACCGT
GGACTTCTACGTGATTACCTACGGCGAGACAGGCGGATACGCC
TGGGCTTATCAGGAGTTCAAGGTGCCTGGATCTAAAAGCACCG
CCACAATCTCCGGCCTGAGCCCCGGCGTGGATTACACCATCAC
CGTCTACGCCTACCAGGTGATCGGCGCCATCTGGAAGTACTAC
AGCCCAATCAGCATCAACTACAGAACCTAA

This polynucleotide can be coupled to any of a variety of promoter and enhancer sequences, as well as 3′ polyadenylation sequences, which are operable in human cells.

In one embodiment isolated polynucleotides of the present disclosure include those encoding the binding molecules described supra. Exemplary isolated polynucleotide molecules include those encoding a FN3 domain comprising one or more of a BC loop amino acid sequence of SEQ ID NO: 3, an CD loop amino acid sequence of SEQ ID NO: 7, a DE loop amino acid sequence of SEQ ID NO: 9, and an FG loop amino acid sequence of SEQ ID NO: 11. In some embodiments, the FN domain encoded by the polynucleotide further comprises an amino acid substitution in one or more beta strands as discussed supra, including at one or more residues corresponding to residues Y31, R33, and/or T49 of SEQ ID NO: 1. In some embodiments, the FN3 domain encoded by the polynucleotide of the disclosure comprises an amino acid sequence that is at least 80% identical to an amino acid sequence of SEQ ID NO: 68. In some embodiments, the FN3 domain encoded by the polynucleotide of the present disclosure comprises an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to an amino acid sequence of SEQ ID NO: 68. In some embodiments, the polynucleotide of the present disclosure encodes an FN3 domain comprising an amino acid sequence of SEQ ID NO: 68 (Mb(NRAS_9)).

In one embodiment, the exemplary isolated polynucleotide encoding the FN3 domain of SEQ ID NO: 68 (Mb(NRAS_9)) is a DNA molecule that is codon-optimized for expression in human cells. One such polynucleotide includes the DNA sequence according to SEQ ID NO: 73 as follows:

GTTTCCAGCGTGCCTACCAAGCTGGAAGTGGTGGCCGCCACCC
CTACAAGCCTGCTGATCAGCTGGGACGCCCCTGCTGTGACCGT
GGATTTCTACGTGATTACATACGGCGAGACAGGATCTTATTAC
CCCTACGCCCAGGAGTTCAAGGTGCCAGGCTCTAAAAGCACCG
CCACAATCTCCGGCCTGAAGCCTGGCGTGGACTACACCATCAC
CGTCTACGCCTACCAGGTGATCGGCGCTATCTGGAAGTACTAC
AGCCCCATCAGCATCAACTACAGAACCTAA

This polynucleotide can be coupled to any of a variety of promoter and enhancer sequences, as well as 3′ polyadenylation sequences, which are operable in human cells.

Another aspect of the disclosure is directed to a fusion construct, said fusion construct comprising a first portion, said first portion comprising a polynucleotide encoding the NRAS binding polypeptide as describe herein and a second portion operatively coupled to said first portion, said second portion comprising a polynucleotide encoding a E3 ubiquitin ligase subunit or an imaging protein or peptide thereof.

In any embodiment, the second portion of a NRAS fusion construct is a polynucleotide encoding an E3 ubiquitin ligase subunit. Suitable polynucleotides include those that encode an E3 ubiquitin ligase subunit selected from the group consisting of speckle type POZ protein (SPOP; NP_003930), E3 ubiquitin-protein ligase CHIP (NP_005852), Ankyrin repeat and SOCS box protein 1 (ASB1; NP_001035535), Suppressor of cytokine signaling 2 (SOCS2; NP_003868), DNA damage-binding protein 2 (DDB2; NP_0000098), Protein cereblon (CRBN; NP_057386), von Hippel-Lindau disease tumor suppressor (VHL; NP_000542), S-phase kinase-associated protein 2 (SKP2; NP_005974), F-box/WD repeat-containing protein 7 (FBW7; NP_060785), F-box/WD repeat-containing protein 1A isoform 2 (NP_003930) (see Lim et al., “bioPROTACs as Versatile Modulators of Intracellular Therapeutic Targets Including Proliferating Cell Nuclear Antigen (PCNA),” PNAS 117 (11): 5791-5800 (2020), which is hereby incorporated by reference in its entirety.

In any embodiment, the second portion of a NRAS fusion construct is a polynucleotide encoding an imaging agent. In any embodiment, the imaging agent is a fluorescent or luminescent proteins. Exemplary fluorescent proteins include, without limitation, Aequorea green fluorescent protein and derivatives thereof, Anthozoan fluorescent protein and derivatives thereof, Discosoma red fluorescent protein and derivatives thereof, Anemonia fluorescent protein and derivatives thereof. Exemplary luminescent proteins include, without limitation, Firefly luciferase protein and derivatives thereof, Renilla luciferase protein and derivatives thereof, and bacterial luciferase protein and derivatives thereof.

The polynucleotides of the disclosure may be produced by chemical synthesis such as solid phase polynucleotide synthesis on an automated polynucleotide synthesizer and assembled into complete single or double stranded molecules. Alternatively, the polynucleotides of the disclosure may be produced by other techniques such as solid phase synthesis, PCR followed by routine cloning, as well as in vitro transcription. Techniques for producing or obtaining polynucleotides of a given known sequence are well known in the art.

The polynucleotides described herein may comprise at least one non-coding sequence, such as a promoter or enhancer sequence, intron, polyadenylation signal, a cis sequence facilitating RepA binding, and the like. The polynucleotide sequences may also comprise additional sequences encoding additional amino acids that encode for example a marker or a tag sequence such as a histidine tag or an HA tag to facilitate purification or detection of the protein, a signal sequence, a fusion protein partner such as RepA, Fc, bacteriophage coat protein such as pIX or pIII, or yeast protein Aga2.

Exemplary constitutive promoter sequences operable in human cells include, without limitation, an EF1 alpha promoter, for example the EF1 alphaS promoter; the PGK promoter; the CMV or SV40 viral promoters; the GAG promoter; the UBC promoter. Other constitutive promoters can also be used (see Qin et al., “Systematic Comparison of Constitutive Promoters and the Doxycycline-Inducible Promoter,” PLOS One 5 (5): e10611 (2010), which is hereby incorporated by reference in its entirety). The constitutive promoters can be rendered inducible using, e.g., a transcriptional suppression domain (tTS) adjacent to a high-affinity tTS-binding site (tetO), such that expression is suppressed in the absence of doxycycline but restored in the presence of doxycycline.

Another embodiment of the disclosure is a vector comprising at least one or more of the polynucleotides and fusion constructs as described herein. Such vectors may be plasmid vectors, viral vectors, vectors for baculovirus expression, transposon based vectors or any other vector suitable for introduction of the polynucleotides of the invention into a given organism or genetic background by any means. Such vectors may be expression vectors comprising nucleic acid sequence elements that can control, regulate, cause or permit expression of a polypeptide encoded by such a vector. Such elements may comprise transcriptional enhancer binding sites, RNA polymerase initiation sites, ribosome binding sites, and other sites that facilitate the expression of encoded polypeptides in a given expression system. Such expression systems may be cell-based, or cell-free systems well known in the art.

In any embodiment, the vector comprising the polynucleotide encoding the NRAS binding polypeptide or fusion construct is a viral vector. Suitable viral vectors include, without limitation, lentiviral vector, an adeno-associated viral vector, vaccinia vector, and a retroviral vector.

Another embodiment of the present disclosure is a host cell comprising the above-described vectors. The NRAS binding molecules and/or NRAS binding peptide conjugates disclosed herein can be optionally produced by a cell line, a mixed cell line, an immortalized cell or clonal population of immortalized cells, as well known in the art (see e.g., Ausubel et al., ed., Current Protocols in Molecular Biology, John Wiley & Sons, Inc., NY, N.Y. (1987-2001); Sambrook et al., Molecular Cloning: A Laboratory Manual, 2nd Edition, Cold Spring Harbor, N.Y. (1989); Harlow and Lane, Antibodies, a Laboratory Manual, Cold Spring Harbor, N.Y. (1989); Colligan et al., eds., Current Protocols in Immunology, John Wiley & Sons, Inc., NY (1994-2001); Colligan et al., Current Protocols in Protein Science, John Wiley & Sons, NY, N.Y., (1997-2001), which are hereby incorporated by reference in their entirety).

The host cell chosen for expression may be of mammalian origin or may be selected from COS-1, COS-7, HEK293, BHK21, CHO, BSC-1, He G2, SP2/0, HeLa, myeloma, lymphoma, yeast, insect or plant cells, or any derivative, immortalized or transformed cell thereof. Alternatively, the host cell may be selected from a species or organism incapable of glycosylating polypeptides, e.g., a prokaryotic cell or organism, such as BL21, BL21 (DE3), BL21-GOLD (DE3), XL1-Blue, JM109, HMS174, HMS174 (DE3), and any of the natural or engineered E. coli spp, Klebsiella spp., or Pseudomonas spp strains.

Mammalian expression systems are generally the preferred platform for manufacturing biopharmaceuticals, as these cell lines are able to produce large, complex proteins with post-translational modifications similar to those produced in humans. Moreover, in the case of mammalian cell lines, most proteins can be secreted rather than requiring cell lysis to extract with subsequent protein refolding (as is the case with bacteria/prokaryotes).

Human cell lines have the ability to produce proteins most similar to those synthesized naturally in humans, which may be an advantage compared with other mammalian expression systems. In particular, the structure, number and location of post-translational N-glycans can affect the biologic activity, protein stability, clearance rate and immunogenicity of biotherapeutic proteins. Exemplary human cell lines that can be used to recombinantly express the NRAS binding molecules include, without limitation, HEK293, fibrosarcoma HT-1080, PER.C6, HKB-11, CAP, and HuH-7 human cell lines.

As noted above, the NRAS binding molecules and/or NRAS binding peptide conjugates, when recombinantly expressed, can then be purified using routine techniques that are well known in the art.

### Pharmaceutical Compositions & Therapeutic Uses

A further aspect of the invention relates to methods of using the products of the present invention, including the NRAS binding peptides, NRAS binding peptide conjugates, and polynucleotides encoding the same, as well as pharmaceutical compositions that contain the peptides, conjugates, or polynucleotides of the invention. In accordance with one embodiment of the invention, it is particularly desirable that the NRAS binding peptide is highly selective, and preferably displays no significant binding to wildtype (normal) KRAS and HRAS, such that the administration step does not interfere with normal KRAS or HRAS-mediated signaling or cellular function.

The NRAS binding peptides, NRAS binding peptide conjugates, and polynucleotides encoding the same are advantageously administered as pharmaceutical compositions comprising an active therapeutic agent (i.e., the NRAS binding peptides, conjugates, and polynucleotides) and one or more of a variety of other pharmaceutically acceptable components. See REMINGTON: THE SCIENCE AND PRACTICE OF PHARMACY (21^{st} Edition) (2005) (Troy, D. B. et al. (Eds.) Lippincott Williams & Wilkins (Publs.), Baltimore MD), which is hereby incorporated by reference in its entirety. The preferred form depends on the intended mode of administration and therapeutic application. The compositions can also include, depending on the formulation desired, pharmaceutically acceptable, non-toxic carriers, excipients, diluents, fillers, salts, buffers, detergents (e.g., a nonionic detergent, such as Tween®20 or Tween®80), stabilizers (e.g., sugars or protein-free amino acids), preservatives, tissue fixatives, solubilizers, and/or other materials suitable for inclusion in a pharmaceutical composition, and which are vehicles commonly used to formulate pharmaceutical compositions for animal or human administration. The diluent is selected to not affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, or non-toxic, nontherapeutic, non-immunogenic stabilizers and the like. Examples of suitable aqueous and non-aqueous carriers which may be employed in the pharmaceutical compositions of the present invention include water, saline, phosphate-buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well-known in the pharmaceutical arts.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the present invention is contemplated.

The compositions may also include large, slowly metabolized macromolecules, such as proteins, polysaccharides like chitosan, polylactic acids, polyglycolic acids and copolymers (e.g., latex functionalized sepharose, agarose, cellulose, and the like), polymeric amino acids, amino acid copolymers, and lipid aggregates (e.g., oil droplets or liposomes). Suitability for carriers and other components of pharmaceutical compositions is determined based on the lack of significant negative impact on the desired biological properties of the NRAS binding polypeptide of the present invention (e.g., less than a substantial impact (e.g., 10% or less relative inhibition, 5% or less relative inhibition, etc.) on binding to NRAS.

The pharmaceutical compositions of the present invention may also comprise pharmaceutically acceptable antioxidants for instance (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutical compositions of the present invention may also comprise isotonicity agents, such as sugars, polyalcohols, such as mannitol, sorbitol, glycerol or sodium chloride in the compositions.

The pharmaceutical compositions of the present invention may also contain one or more additives appropriate for the chosen route of administration such as preservatives, wetting agents, emulsifying agents, dispersing agents, preservatives or buffers, which may enhance the shelf life or effectiveness of the pharmaceutical composition. The compounds of the present invention may be prepared with carriers that will protect the polypeptides, conjugates, or polynucleotides against rapid release, such as a controlled release formulation, including implants, transdermal patches, and microencapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, polyorthoesters, and polylactic acid alone or with a wax, or other materials well-known in the art. Methods for the preparation of such formulations are generally known to those skilled in the art. See, e.g., SUSTAINED AND CONTROLLED RELEASE DRUG DELIVERY SYSTEMS, J. R. Robinson, ed., Marcel Dekker, Inc., New York, 1978.

In one embodiment, the compounds of the present invention may be formulated to ensure proper distribution in vivo. Pharmaceutically acceptable carriers for parenteral administration include sterile aqueous solutions or dispersions and sterile powders for the extemporaneous preparation of sterile injectable solutions or dispersion. The use of such media and agents for pharmaceutically active substances is known in the art.

Pharmaceutical compositions for injection must typically be sterile and stable under the conditions of manufacture and storage. The composition may be formulated as a solution, microemulsion, liposome, or other ordered structure suitable to achieve high drug concentration. The carrier may be an aqueous or non-aqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, by the use of a coating such as lecithin, by the maintenance of the required particle size in the case of dispersion and by the use of surfactants. In many cases, it will be preferable to include isotonic agents, for example, sugars, polyalcohols such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the active compound in the required amount in an appropriate solvent with one or a combination of ingredients e.g., as enumerated above, as required, followed by sterilization microfiltration. Generally, dispersions are prepared by incorporating the active compound into a sterile vehicle that contains a basic dispersion medium and the required other ingredients. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

For parenteral administration, agents of the present invention are typically formulated as injectable dosages of a solution or suspension of the substance in a physiologically acceptable diluent with a pharmaceutical carrier that can be a sterile liquid such as water, oil, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin. Peanut oil, soybean oil, and mineral oil are all examples of useful materials. In general, glycols, such as propylene glycol or polyethylene glycol, are preferred liquid carriers, particularly for injectable solutions. Agents of the invention can be administered in the form of a depot injection or implant preparation which can be formulated in such a manner as to permit a sustained release of the active ingredient. An exemplary composition comprises an NRAS binding peptide at about 5 mg/mL, formulated in aqueous buffer consisting of 50 mM L-histidine, 150 mM NaCl, adjusted to pH 6.0 with HCl.

Typically, compositions are thus prepared as injectables, either as liquid solutions or suspensions; solid forms suitable for solution in, or suspension in, liquid vehicles prior to injection can also be prepared. The preparation also can be emulsified or encapsulated in liposomes or micro particles, such as polylactide, polyglycolide, or copolymer, for enhanced adjuvant effect (Langer, et al., Science 249:1527 (1990); Hanes, et al., Advanced Drug Delivery Reviews 28:97-119 (1997), which are hereby incorporated by reference in their entirety). Additional formulations suitable for other modes of administration include oral, intranasal, and pulmonary formulations, suppositories, and transdermal applications.

One such method of use is for the treatment of cancer in a subject, particularly the treatment of an NRAS-mediated cancer. Exemplary cancers that can be treated include, without limitation, those where oncogenic mutant NRAS forms are implicated, which includes colon cancer, rectal cancer, follicular thyroid cancer, melanoma, leukemia, and myeloma. In carrying out this method, a subject is administered an amount of the NRAS binding peptide, conjugate, or polynucleotide encoding the same, or a pharmaceutical composition containing the same, which is effective to treat the cancer.

In one embodiment, a pharmaceutical composition of the present invention is administered parenterally. The phrases “parenteral administration” and “administered parenterally” as used herein denote modes of administration other than enteral and topical administration, usually by injection, and include epidermal, intravenous, intramuscular, intraarterial, intrathecal, intracapsular, intracranial, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection, subcutaneous and infusion. In one embodiment that pharmaceutical composition is administered by intravenous or subcutaneous injection or infusion.

Effective doses of the provided NRAS binding peptide or conjugate as described herein, for the treatment of the above-described conditions may vary depending upon many different factors, including means of administration, target site, physiological state of the patient, other medications administered, etc. Treatment dosages are typically titrated to optimize their safety and efficacy. On any given day that a dosage is given, the dosage of the NRAS binding peptide or conjugate as described herein may range from about 0.0001 to about 100 mg/kg, and more usually from about 0.01 to about 5 mg/kg, of the patient's body weight. For example, dosages can be 1 mg/kg body weight or 10 mg/kg body weight or within the range of 1-10 mg/kg body weight. Exemplary dosages thus include: from about 0.1 to about 10 mg/kg body weight, from about 0.1 to about 5 mg/kg body weight, from about 0.1 to about 2 mg/kg body weight, from about 0.1 to about 1 mg/kg body weight, for instance about 0.15 mg/kg body weight, about 0.2 mg/kg body weight, about 0.5 mg/kg body weight, about 1 mg/kg body weight, about 1.5 mg/kg body weight, about 2 mg/kg body weight, about 5 mg/kg body weight, or about 10 mg/kg body weight.

A physician or veterinarian having ordinary skill in the art may readily determine and prescribe the effective amount of the NRAS binding peptide or conjugate (in the form of a pharmaceutical composition), as required. For example, the physician or veterinarian could start doses of the inventive pharmaceutical composition at levels lower than that required in order to achieve the desired therapeutic effect and gradually increase the dosage until the desired effect is achieved. In general, a suitable daily dose of a composition of the present invention will be that amount of the NRAS binding peptide or conjugate which is the lowest dose effective to produce a therapeutic effect. Such an effective dose will generally depend upon the factors described above. Administration may e.g. be intravenous, intramuscular, intraperitoneal, or subcutaneous, and for instance administered proximal to the site of the target. If desired, the effective daily dose of a pharmaceutical composition may be administered as two, three, four, five, six or more sub-doses administered separately at appropriate intervals throughout the day, optionally, in unit dosage forms. While it is possible the NRAS binding peptide or conjugate of the present invention to be administered alone, it is preferable to administer the NRAS binding peptide or conjugate as a pharmaceutical composition as described above.

In accordance with another embodiment of the invention, where the NRAS binding peptide conjugate includes an imaging agent such as a fluorescent polypeptide or contrasting agent, the conjugate can be used for imaging tissue samples in which mutant NRAS-mediated cancer cells are present or tumor sites where mutant NRAS-mediated cancer cells reside.

The above-identified pharmaceutical compositions containing the NRAS binding peptide conjugate can be introduced to a tissue sample and used for visual identification of mutant NRAS-mediated cancer cells in the sample. This manner of in vitro identification can be carried out using standard tissue sample preparation including analysis of fresh or frozen tissue samples, as well as histological tissue samples. NRAS-mediated cancer cells can be identified by their decoration with the fluorescent polypeptide (as a component of the inventive conjugate).

For in vivo imaging, NRAS binding peptide conjugates can be used for labeling of surface bound mutant NRAS at tumor sites. In this aspect, the conjugates include a suitable contrasting agent. Superparamagnetic contrast agents have greater magnetic susceptibility than traditional MRI contrast agents (e.g., gadolinium) and some are commercially available, such as suspensions of polymer-coated ferromagnetic nanoparticles in water (Alexiou et al, J. Nanosci. Nanotechnol. 6:2762-2768 (2006), which is hereby incorporated by reference in its entirety). Their presence significantly weakens the MRI signal and creates a negative enhancement effect on images. MRI-enhancing contrast agents include superparamagnetic iron oxide (SPIO, >50 nm) and ultrasmall superparamagnetic iron oxide (USPIO, <50 nm) particles (Couvreur and Vauthier, Pharmaceutical Research 23:1417 (2006), which is hereby incorporated by reference in its entirety). Sequestration of SPIO nanoparticles by the reticulo-endothelial system provides high contrast imaging of splenic/hepatic tumors and metastases. USPIO nanoparticles have longer circulation times in the blood and broader tissue distribution because they avoid reticuloendothelial system sequestration; they are ideal for detecting metastases in lymph nodes. The present invention presents an entirely novel approach for accumulation of the magnetic particles on tumor cell surfaces. U.S. Pat. No. 5,869,023 to Ericcson, which is hereby incorporated by reference in its entirety, describes a method for magnetic resonance imaging using positive and negative contrasting agents. The same approach can be used with the products of the present invention as contrast agents.

### Nontherapeutic Uses

The NRAS binding peptides of the invention can also be used in a method for screening small molecule inhibitors that are active at the same binding site on NRAS. The set up for this assay can be an optical-based detection system such as that described in Quevedo et al., Nature Comm. 9:3169, doi: 10.1038/s41467-018-05707-2 (2018), which is hereby incorporated by reference in its entirety. Briefly, Quevedo reports using a surface-plasmon resonance detection procedure to screen a small-molecule library for the ability of small molecules to competitively displace an anti-mutant RAS antibody fragment. Any other optical detection can similarly be used to assess displacement of, in this case, the NRAS binding peptides from NRAS. Based on the low nanomolar affinity of the NRAS binding peptide for NRAS, it is expected that such a screening assay will yield small molecules inhibitors of NRAS that bind to the region of NRAS recognized by the NRAS binding polypeptide.

It should be appreciated, however, that any suitable detection system can be utilized, and the invention is not limited to the use of surface plasmon resonance optical detection systems. Thus, any detection system that is capable of measuring a signal change associated with displacement of the NRAS binding polypeptide from NRAS can be utilized. These detection systems can be label-free optical detection systems or label-based detection systems. The NRAS binding peptides may include a label, such as a fluorescent protein conjugate, which when displaced will cause a reduction in the fluorescence. Other label-based detection systems include florescence spectrometer, fluorescence microscopy, and flow cytometry, and AlphaScreen (Perkin Elmer). The method can utilize purified samples of NRAS and NRAS binding polypeptide or utilize cells in which NRAS and NRAS binding polypeptide, optionally with suitable labels (e.g., fluorescent protein conjugates), are expressed. Label-free optical detection systems may measure any one or more of the intensity of reflected light, the angular shift in reflectivity over time, absorption, or interferometric pattern. Other label-free detection systems include, without limitation, interferometry detection systems and evanescent wave detection systems.

### EXAMPLES

The examples below are intended to exemplify the practice of embodiments of the disclosure but are by no means intended to limit the scope thereof.

### Example 1—Development of Monobodies Selective to Human NRAS

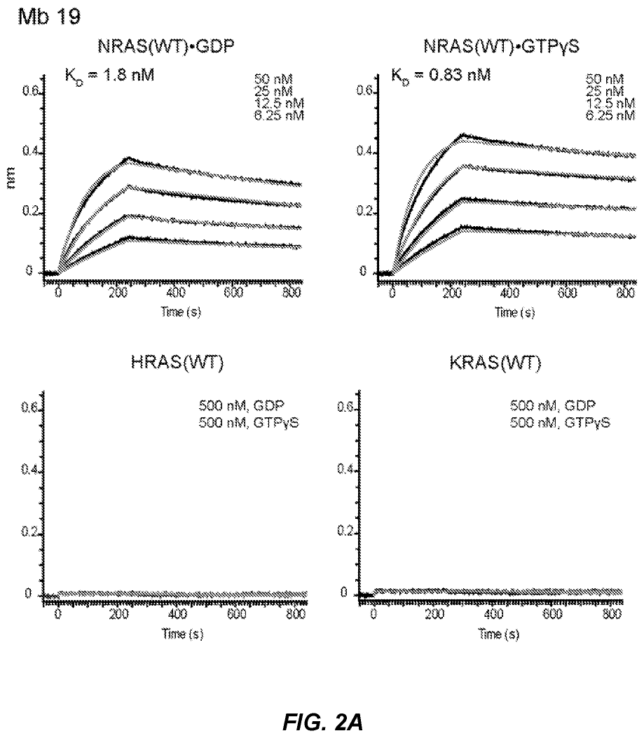

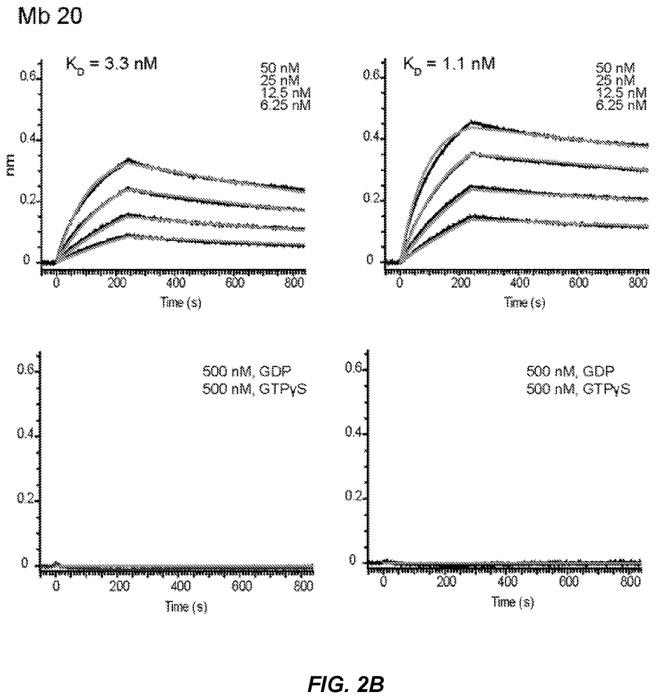

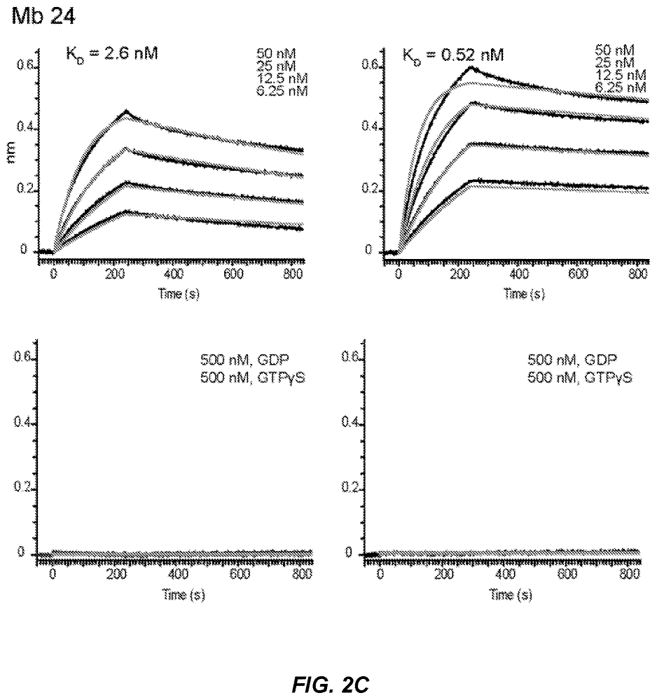

Previously established methods for monobody development (Spencer-Smith et al., “Inhibition of RAS Function Through Targeting an Allosteric Regulatory Site,” Nat Chem Biol. 13 (1): 62-68 (2017) (doi: 10.1038/nchembio.2231; PMID: 27820802); Koide et al., “Teaching an Old Scaffold New Tricks: Monobodies Constructed Using Alternative Surfaces of the FN3 Scaffold,” J Mol Biol. 415:393-405 (2012) (doi: 10.1016/j.jmb.2011.12.019; PMID: 22198408); Teng et al., “Selective and Noncovalent Targeting of RAS Mutants for Inhibition and Degradation,” Nat Commun. 12 (1): 2656 (2021) (doi: 10.1038/s41467-021-22969-5; PMID: 33976200), each of which is hereby incorporated by reference in its entirety) were employed using NRAS as a target and KRAS as a negative selection target. As a result, three clones were identified that show selective binding to NRAS over KRAS and HRAS-Mb(NRAS_19), SEQ ID NO: 12; Mb(NRAS_20), SEQ ID NO: 13; and Mb(NRAS_24), SEQ ID NO: 14 (FIG. 1). These monobodies were produced as purified proteins and their binding characteristics against purified RAS proteins were analyzed using biolayer interferometry (BLI) (FIGS. 2A-2C). These results show that the three monobodies have high affinity and exquisite selectivity to NRAS in both GTPYS- and GDP-bound forms. The insensitivity of these monobodies to the nucleotide state is similar to NS1 (Spencer-Smith et al., “Inhibition of RAS Function Through Targeting an Allosteric Regulatory Site,” Nat Chem Biol. 13 (1): 62-68 (2017) (doi: 10.1038/nchembio.2231; PMID: 27820802), which is hereby incorporated by reference in its entirety).

### Example 2—NRAS-Selective Monobodies Bind to Epitopes that Overlap with the Epitope of NS1

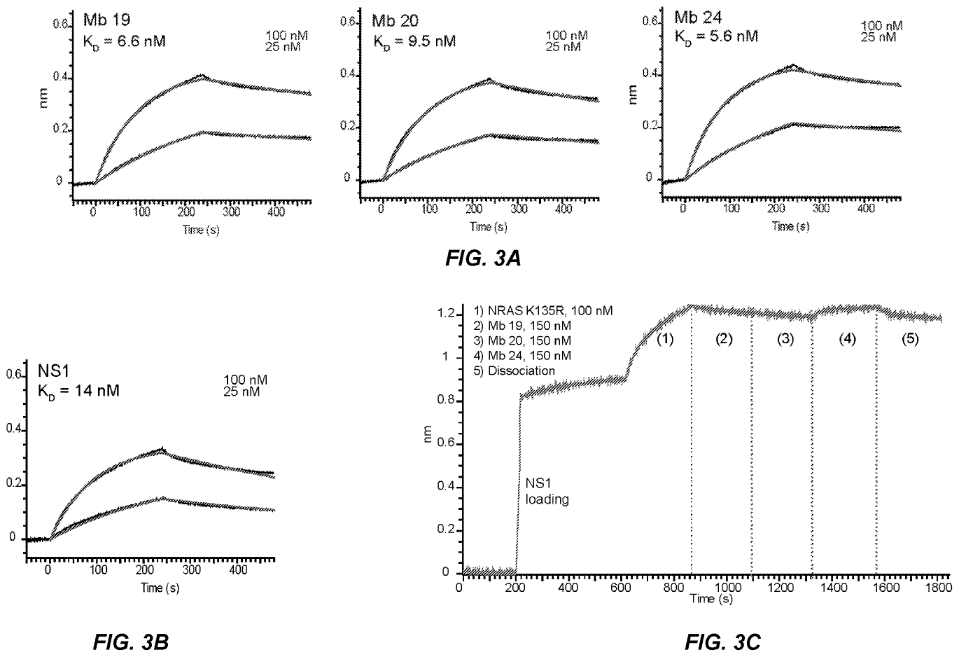

To test whether these new monobodies bind to an epitope in NRAS that is similar to the epitope of NS1 in HRAS, an NRAS mutant was first developed that has an H/KRAS-mimicking mutation, K135R. The new NRAS-selective monobodies retained their binding to NRAS (K135R) (FIGS. 3A-3B). NS1 gained binding to NRAS (K135R), supporting the importance of R135 in the binding of NS1 to HRAS and KRAS as previously reported (Spencer-Smith et al., “Inhibition of RAS Function Through Targeting an Allosteric Regulatory Site,” Nat Chem Biol. 13 (1): 62-68 (2017) (doi: 10.1038/nchembio.2231; PMID: 27820802), which is hereby incorporated by reference in its entirety). NS1 was found to inhibit the binding of the NRAS-selective monobodies to NRAS (K135R). Because NS1 does not cause structural changes in regions in HRAS distant to its epitope (Spencer-Smith et al., “Inhibition of RAS Function Through Targeting an Allosteric Regulatory Site,” Nat Chem Biol. 13 (1): 62-68 (2017) (doi: 10.1038/nchembio.2231; PMID: 27820802), which is hereby incorporated by reference in its entirety), it is unlikely that the observed inhibitory effect of NS1 is mediated by long-range allosteric effect. Thus, these results demonstrate that these monobodies bind to a region that overlaps with the epitope of NS1, the alpha4-alpha5 region (Spencer-Smith et al., “Inhibition of RAS Function Through Targeting an Allosteric Regulatory Site,” Nat Chem Biol. 13 (1): 62-68 (2017) (doi: 10.1038/nchembio.2231; PMID: 27820802), which is hereby incorporated by reference in its entirety). This conclusion, in turn, strongly supports the expectation that these monobodies will inhibit NRAS-mediated signaling when they are introduced in cells.

### Example 3—Mutagenesis Study of Mb(NRAS_24) (SEQ ID NO: 14)

Scanning mutagenesis of the FG loop sequence of Mb(NRAS_24) (SEQ ID NO: 14) was performed to define permissible mutations. One or two residues in the sequence were mutated and a yeast display library was constructed. Yeast cells showing binding to NRAS (WT) were recovered and the sequences of encoded monobodies were determined. This analysis identified mutations listed in FIGS. 4A-4B.

Although preferred embodiments have been depicted and described in detail herein, it will be apparent to those skilled in the relevant art that various modifications, additions, substitutions, and the like can be made without departing from the spirit of the application and these are therefore considered to be within the scope of the application as defined in the claims which follow.

What is claimed:

1. A Neuroblastoma-RAS (“NRAS”) binding polypeptide, said binding polypeptide comprising a fibronectin type III (FN3) domain having one or more modified loop sequences comprising a modified FG loop amino acid sequence, a modified BC loop amino acid sequence, a modified CD loop amino acid sequence, a modified DE loop amino acid sequence, or a combination thereof, and optionally one or more modified beta strand sequences, wherein said one or more modified loop sequences and said one or more optional modified beta strand sequences enable selectively binding to NRAS but not KRAS or HRAS.

2. The NRAS binding polypeptide of claim 1, wherein said binding polypeptide binds to NRAS in an active GTP-bound state or in the GDP-bound state.

3. The NRAS binding polypeptide of claim 1 or claim 2, wherein said binding polypeptide selectively binds NRAS with an affinity (K_{D}) of less than about 100 nM.

4. The NRAS binding polypeptide of claim 1 or claim 2, wherein said binding polypeptide selectively binds NRAS with an affinity (K_{D}) of less than about 10 nM.

5. The binding polypeptide of any one of claims 1-4, wherein the FG loop amino acid sequence comprises the amino acid sequence of SEQ ID NO: 10 or 11.

6. The binding polypeptide of any one of claims 1-4, wherein the FG loop amino acid sequence is selected from any one of SEQ ID NOS: 12-64.

7. The binding polypeptide of any one of claims 1-6, wherein the CD loop amino acid sequence comprises the amino acid sequence of SEQ ID NO: 4.

8. The binding polypeptide of any one of claims 1-6, wherein the modified CD loop amino acid sequence comprises one of SEQ ID NOS: 5-7.

9. The binding polypeptide of any one of claims 1-8, wherein the BC loop amino acid sequence comprises the amino acid sequence SEQ ID NO: 2 or 3.

10. The binding polypeptide of any one of claims 1-8, wherein the DE loop amino acid sequence comprises the amino acid sequence SEQ ID NO: 8 or 9.

11. The binding polypeptide of any one of claims 1-10, wherein the FN3 domain is a human fibronectin type III tenth domain (¹⁰Fn3) of SEQ ID NO: 1 comprising the at least one modified loop amino acid sequence.

12. The binding polypeptide of claim 11, wherein the one or more modified beta strands in the ¹⁰Fn3 domain comprises an amino acid substitution in one of the C, D, F, and G beta-strands, or a combination thereof.

13. The binding polypeptide of claim 11, wherein the amino acid substitution is at one or more residues selected from Y31 and R33 of SEQ ID NO: 1.

14. The binding polypeptide of claim 13, wherein the amino acid substitution at Y31 is Y31F.

15. The binding polypeptide of claim 13, wherein the amino acid substitution at R33 is R33V.

16. The binding polypeptide of claim 11 or 12, wherein the amino acid substitution is at one or more residues selected from E47 and T49 of SEQ ID NO: 1.

17. The binding polypeptide of claim 16, wherein the amino acid substitution at T49 is T49K.

18. The binding polypeptide of any one of claims 1-17, wherein the FN3 domain comprises:
a CD loop amino acid sequence of SEQ ID NO: 4, an FG loop amino acid sequence of SEQ ID NO: 10, a BC loop amino acid sequence of SEQ ID NO: 3, and a DE loop amino acid sequence of SEQ ID NO: 9 (Consensus).

19. The binding polypeptide of any one of claims 1-18, wherein the FN3 domain comprises:
(i) a CD loop amino acid sequence of SEQ ID NO: 5, an FG loop amino acid sequence of SEQ ID NO: 11, a BC loop amino acid sequence of SEQ ID NO: 3, and a DE loop amino acid sequence of SEQ ID NO: 9 (NRAS_19); or
(ii) a CD loop amino acid sequence of SEQ ID NO: 6, an FG loop amino acid sequence of SEQ ID NO: 11, a BC loop amino acid sequence of SEQ ID NO: 3, and a DE loop amino acid sequence of SEQ ID NO: 9 (NRAS_20); or
(iii) a CD loop amino acid sequence of SEQ ID NO: 7, an FG loop amino acid sequence of SEQ ID NO: 11, a BC loop amino acid sequence of SEQ ID NO: 3, and a DE loop amino acid sequence of SEQ ID NO: 9 (NRAS_24).

20. The binding polypeptide of any one of claims 1-19, wherein the FN3 domain comprises an amino acid sequence that is at least 80% identical to an amino acid sequence selected from the group consisting of SEQ ID NOs: 12-14.

21. The binding polypeptide of any one of claims 1-19, wherein the FN3 domain comprises an amino acid sequence that is at least 90% identical to an amino acid sequence selected from the group consisting of SEQ ID NOs: 12-14.

22. The binding polypeptide of any one of claims 1-19, wherein the FN3 domain comprises an amino acid sequence that is at least 95% identical to an amino acid sequence selected from the group consisting of SEQ ID NOs: 12-14.

23. The binding polypeptide of any one of claims 1-19, wherein the FN3 domain comprises an amino acid sequence selected from the group consisting of SEQ ID NOs: 12-14.

24. The binding polypeptide of any one of claims 1-19, wherein the FN3 domain comprises a variant of the amino acid sequence of SEQ ID NO: 14 that includes one or more of the FG domains according to one of SEQ ID NOS: 15-67.

25. A NRAS binding polypeptide conjugate, said conjugate comprising:
a first portion, said first portion comprising the NRAS binding polypeptide of any one of claims 1-24; and
a second portion coupled to said first portion, said second portion selected from the group consisting of an E3 ubiquitin ligase ligand or an E3 ligase subunit.

26. The NRAS binding polypeptide conjugate of claim 25, wherein the second portion of the conjugate comprises an E3 ubiquitin ligase ligand.

27. The NRAS binding polypeptide conjugate of claim 26, wherein the E3 ubiquitin ligase ligand is a small molecule ligand for an E3 ubiquitin ligase selected from the group consisting of cereblon, Von Hippel-Lindau (VHL), Inhibitor of Apoptosis (IAP), MDM2, RNF114, DCAF16, DCAF15, Kelch-like ECH-associated protein 1 (KEAP1), FEMIB, and aryl hydrocarbon receptor.

28. The NRAS binding polypeptide conjugate of claim 25, wherein the E3 ligase subunit is selected from the group consisting of SPOP (speckle type POZ protein), E3 ubiquitin-protein ligase CHIP, Ankyrin repeat and SOCS box protein 1 (ASB1), Suppressor of cytokine signaling 2 (SOCS2), DNA damage-binding protein 2 (DDB2), Protein cereblon (CRBN), von Hippel-Lindau disease tumor suppressor (VHL), S-phase kinase-associated protein 2 (SKP2), F-box/WD repeat-containing protein 7 (FBW7), and F-box/WD repeat-containing protein 1A isoform 2.

29. The NRAS binding polypeptide conjugate of claim 25 further comprising a delivery vehicle linked to one or both of the first and second portions.

30. The NRAS binding polypeptide conjugate of claim 29, wherein the delivery vehicle comprises a toxin.

31. The NRAS binding polypeptide conjugate of claim 30, wherein the toxin is an attenuated toxin.

32. The NRAS binding polypeptide conjugate of claim 31, wherein the attenuated toxin is an attenuated anthrax toxin, an attenuated diphtheria toxin, attenuated Shiga and Shiga-like toxin.

33. The NRAS binding polypeptide conjugate of claim 29, wherein the delivery vehicle comprises an antibody or binding fragment thereof, or a synthetic binding molecule that binds a cell surface molecule.

34. The NRAS binding polypeptide conjugate of claim 25, wherein the cell surface molecule is a cancer cell specific surface molecule selected from CUB domain-containing protein 1 (CDCP1), Intercellular adhesion molecule 1 (ICAM1), Integrin beta-5 (ITGB5), (5′-nucleotidase) NT5E, Tumor necrosis factor receptor superfamily member 3 (LTBR), Complement decay-accelerating factor (CD55), Aminopeptidase N (ANPEP), CD79, Trophoblast glycoprotein (TPBG), Integrin beta-1 (ITGB1), Prostaglandin F2 receptor negative regulator (PTGFRN), Integrin alpha-5 (ITGA5), and Exosome complex protein LRP1 (LRP1).

35. The NRAS binding polypeptide conjugate of claim 29, wherein the delivery vehicle is a cell penetrating ligand.

36. The NRAS binding polypeptide conjugate of claim 35, wherein the cell penetrating ligand is selected from TAT, cR10, Pep-1, penetratin, and transportan.

37. The NRAS binding polypeptide conjugate of claim 25, wherein the conjugate is linked to or encapsulated by a delivery vehicle that is selected from a nanoparticle, a polymer-based particle, and a lipid-based particle.

38. A polynucleotide encoding the NRAS binding polypeptide of any one of claims 1-24 or the NRAS binding polypeptide conjugate of claim 25.

39. The polynucleotide of claim 38, wherein said polynucleotide is an mRNA encoding the NRAS binding polypeptide of any one of claims 1-24.

40. A vector comprising the isolated polynucleotide of claim 38 or claim 39.

41. The vector of claim 40, wherein said vector is a viral vector.

42. The vector of claim 41, wherein said viral vector is selected from the group consisting of lentiviral vector, an adeno-associated viral vector, vaccinia vector, and a retroviral vector.

43. A delivery vehicle comprising the polynucleotide of claim 38 or claim 39 or the vector of any one of claims 40-42.

44. The delivery vehicle of claim 43, wherein the delivery vehicle is selected from a nanoparticle, a polymer-based particle, and a lipid-based particle.

45. A fusion construct comprising:
a first portion, said first portion comprising a polynucleotide encoding the NRAS binding polypeptide of any one of claims 1-24; and
a second portion, operatively coupled to said first portion, said second portion comprising a polynucleotide encoding a E3 ligase subunit, or an imaging protein or peptide thereof.

46. The fusion construction of claim 45, wherein the second portion is a polynucleotide encoding an E3 ligase subunit.

47. The fusion construct of claim 46, wherein the polynucleotide encodes an E3 ligase subunit selected from the group consisting of: SPOP (speckle type POZ protein), E3 ubiquitin-protein ligase CHIP, Ankyrin repeat and SOCS box protein 1 (ASB1), Suppressor of cytokine signaling 2 (SOCS2), DNA damage-binding protein 2 (DDB2), Protein cereblon (CRBN), von Hippel-Lindau disease tumor suppressor (VHL), S-phase kinase-associated protein 2 (SKP2), F-box/WD repeat-containing protein 7 (FBW7), and F-box/WD repeat-containing protein 1A isoform 2.

48. The fusion construct of claim 45, wherein the second portion is a polynucleotide encoding an imaging protein or peptide thereof.

49. The fusion construct of claim 48, wherein the imaging protein or peptide is selected from a Aequorea green fluorescent protein and derivatives thereof, Anthozoan fluorescent protein and derivatives thereof, Discosoma red fluorescent protein and derivatives thereof, Anemonia fluorescent protein and derivatives thereof, Firefly luciferase protein and derivatives thereof, Renilla luciferase protein and derivatives thereof, and bacterial luciferase protein and derivatives thereof.

50. A pharmaceutical composition comprising the NRAS binding polypeptide of any one of claims 1-24, the NRAS binding polypeptide conjugate of any one of claims 25-36, the isolated polynucleotide of any one of claims 38-39, the vector of any one of claims 40-42, or the fusion construct of any one claims 43-49, and a pharmaceutical carrier.

51. A method for treating cancer in a subject, said method comprising:
administering, to the subject having cancer, the pharmaceutical composition of claim 50 in an amount effective to treat the cancer.

52. The method of claim 51, wherein the cancer is characterized by cancerous cells having an oncogenic mutation in NRAS.

54. The method of any one of claims 51-53, wherein the cancer is colon cancer, rectal cancer, follicular thyroid cancer, melanoma, leukemia, and myeloma.

55. The method of any one of claims 51-54, wherein said administering does not interfere with normal KRAS or HRAS-mediated signaling or cellular function.

56. A method of screening agents for capability of binding NRAS comprising:
providing an affinity complex comprising NRAS and an NRAS binding polypeptide according to one of claims 1-24 that is non-covalently bound to NRAS; and
exposing the complex to an agent and assessing whether the agent displaces the NRAS binding polypeptide from NRAS.

57. The method according to claim 56, wherein the agent is a small molecule.

58. The method according to claim 56, wherein said assessing is carried out using an optical detection system to measure a change in an optical property upon displacement of the NRAS binding polypeptide from NRAS.

59. The method according to claim 58, wherein the optical property is intensity of reflected light, angular shift in reflectivity over time, absorption, interferometric pattern, or any combinations thereof.

60. The method according to claim 58, wherein the optical detection system is a label-free optical detection system.

61. The method according to claim 58, wherein the optical detection system is a surface plasmon resonance detection system, an interferometry detection system, or an evanescent wave detection system.

62. The method according to claim 56, wherein said assessing is carried out using a label and detection of the label is either positively or inversely related to displacement.
